(19) United States
(12) Patent Application Publication
(10) Pub. No.: US 2019/0289804 A1
(43) Pub. Date: Sep. 26, 2019

(54) METHODS FOR INDUCING POLYPLOIDY IN CANNABIS

(71) Applicant: CANOPY GROWTH CORPORATION, Smith Falls (CA)

(72) Inventors: Ekaterina A. BOUDKO, Smith Falls (CA); Jessica L. PARSONS, Smith Falls (CA)

(21) Appl. No.: 16/357,999

(22) Filed: Mar. 19, 2019

Related U.S. Application Data
(60) Provisional application No. 62/646,036, filed on Mar. 21, 2018.
(60) Provisional application No. 62/681,370, filed on Jun. 6, 2018.
(60) Provisional application No. 62/681,394, filed on Jun. 6, 2018.
(60) Provisional application No. 62/681,405, filed on Jun. 6, 2018.
(60) Provisional application No. 62/781,963, filed on Dec. 19, 2018.

(51) Int. Cl. A01H 1/06; A01H 5/12; A01H 6/28

(52) U.S. Cl. CPC A01H 1/06; A01H 6/28; A01H 5/12

(57) ABSTRACT

The present technology generally relates to a method for inducing polyploidy in a Cannabis plant, the method comprising treating the Cannabis plant or a part thereof with an amount of a dinitroaniline compound effective to induce polyploidy.

### CROSS-REFERENCE TO RELATED APPLICATIONS

This application claims the benefit of and priority to U.S. provisional patent application No. 62/646,036, filed on Mar. 21, 2018; to U.S. provisional patent application No. 62/681,370, filed on Jun. 6, 2018; to U.S. provisional patent application No. 62/681,394, filed on Jun. 6, 2018; to U.S. provisional patent application No. 62/681,405, filed on Jun. 6, 2018; and to U.S. provisional patent application No. 62/781,963, filed on Dec. 19, 2018; the content of all of which is herein incorporated in entirety by reference.

### FIELD OF TECHNOLOGY

The present technology generally relates to methods for inducing polyploidy in a plant and in particular in a Cannabis plant and to polyploid Cannabis plant obtained by the present methods.

### BACKGROUND INFORMATION

Recently, there has been renewed interest in Cannabis due to its many medicinal effects, particularly the treatment of epilepsy, pain, and nausea associated with cancer treatment (Andre et al., 2016; Thomas and Elsohly, 2016). While there are hundreds of different active metabolites present in Cannabis, two cannabinoids are present in high concentrations, and are generally considered to be the most important: Δ⁹-tetrahydrocannabinol (THC) and cannabidiol (CBD). THC is responsible for the well-known psychoactive properties of Cannabis whereas non-intoxicating CBD is widely used for pain, anxiety, depression, and sleep disorders (Andre et al., 2016; Corroon and Phillips, 2018). Another group of important chemicals is the terpenes, which contribute to the aroma and flavour of Cannabis products but also function as active metabolites (Russo, 2011; Andre et al., 2016). All of these metabolites are produced and stored within glandular trichomes that mainly develop on the inflorescence of the plant (Marks et al., 2009; Andre et al., 2016).

Several medicinal cannabinoid preparations are available. However, using whole Cannabis can be more effective than the single ingredient preparations for some conditions due to the synergy between multiple phytochemicals. In particular, CBD and the terpenes can modulate the effects of THC (Wilkinson et al., 2003; Brenneisen, 2007; Russo, 2011; Andre et al., 2016). Therefore, developing a wider variety of Cannabis strains may be preferable to new formulations of the active ingredients. Historically, new Cannabis strains have been developed through conventional breeding methods. However, these methods can be imprecise, and require several generations before the desired traits are obtained and a stable strain is produced.

One strategy to accelerate breeding development is a chromosome doubling event called polyploidization (Sattler et al., 2016). Polyploidization is common in the plant kingdom and has been associated with increased genetic diversity in some plant lineages (Comai, 2005). Desirable consequences of polyploidy for plant breeding include the buffering of deleterious mutations, increased heterozygosity, and hybrid vigor (Sattler et al., 2016). Consequently, polyploids often have phenotypic traits that are distinct from diploids, including larger flowers or leaves (Dermen, 1940; Rêgo et al., 2011; Trojak-Goluch and Skomra, 2013; Sattler et al., 2016; Talebi et al., 2017). Increases in active metabolite concentration in tetraploids are reported for numerous medicinal plants including Artemisia annua (Wallaart et al., 1999), Papaver somniferum (Mishra et al., 2010), Datura stramonium (Berkov and Philipov, 2002), Thymus persicus (Tavan et al., 2015), Echinacea purpurea (Abdoli et al., 2013) and Tanacetum parthenium (Majdi et al., 2010). Currently, chemical mitotic inhibitory agents such as colchicine or dinitroanilines are used to induce polyploidy in crop plants. A typical example is the production of tetraploid watermelon plants for the production of seedless triploid watermelon (Compton et al., 1996).

The introduction of some of these polyploid traits would be beneficial for the cultivation of Cannabis. Cannabis is diploid plant with 20 chromosomes (van Bakel et al., 2011). Doubling the chromosome set should allow more flexibility to increase potency or tailor the cannabinoid ratios. A handful of studies support the theory that polyploid Cannabis might have higher potency, although the results are mixed, with some studies finding decreases in THC (Clarke, 1981; Bagheri and Mansouri, 2015; Mansouri and Bagheri, 2017). However, these studies were conducted with hemp.

Previously, researchers have used colchicine to induce Cannabis polyploids. For example, colchicine solution was dripped onto seedling tips several times a day. However, this resulted in a difficult and time-consuming method and, due to lack of stability from the seed; there was no baseline for directly comparing diploids to generated polyploids.

Polyploidy can be induced through application of antimitotic agents to seeds, seedlings, in vivo shoot tips, or in vitro explants (Dermen, 1940; Petersen et al., 2003; Talebi et al., 2017). However, drug-type Cannabis strains are not genetically stable when propagated through seeds, and while there has been little success in regenerating Cannabis shoots from callus, the propagation of high THC drug-type Cannabis in tissue culture using nodal explants has been described. These plants have been shown to be genetically and chemically stable through 30 rounds of tissue culture propagation (Lata et al., 2009; Lata et al., 2016).

As such, there remains a need in the field of technology for improved methods and improved techniques for inducing polyploidy in Cannabis that alleviate at least some of these drawbacks.

### SUMMARY OF DISCLOSURE

Without wishing to be bound to any specific theory, embodiments of the present technology have been developed based on the advancements by the present developers of techniques for inducing polyploidy in Cannabis plant, in particular in Cannabis sativa. The present developers have comprehended that dinitroanilines induce polyploidy in Cannabis. As such and broadly speaking, embodiments of the present technology contemplate using dinitroanilines to induce polyploidy in a Cannabis plant and in particular in Cannabis sativa.

Other aspects and features of the present disclosure will become apparent to those ordinarily skilled in the art upon review of the following description of specific embodiments in conjunction with the accompanying drawings.

### BRIEF DESCRIPTION OF THE DRAWINGS

All features of embodiments which are described in this disclosure are not mutually exclusive and can be combined with one another. For example, elements of one embodiment can be utilized in the other embodiments without further mention. A detailed description of specific embodiments is provided herein below with reference to the accompanying drawings in which:

FIG. 1 is a schematic representation of how endopolyploidy occurs naturally in some of the cells of a plant;

FIG. 2 is a photograph of Cannabis sativa wherein the arrows indicate the position of axillary buds on Cannabis sativa;

FIG. 3 is a flow diagram of a method for producing a polyploid plant of Cannabis sativa according to one embodiment of the present technology;

FIG. 4 is a photograph of chromosomes prepared using the root tip squash method from root tip cells of Hindu Kush Cannabis sativa, wherein individual chromosomes have been outlined;

FIG. 5 is a photograph of rooted axillary bud explants undergoing acclimatization (hardening off) for up to 4 weeks in soil; Panels A and B are immediately after transplant; Panels C and D are after 1 month in soil;

FIG. 6 is a photograph of prophase chromosomes in the root tip cells of various Cannabis sativa genotypes; Left to right: Cannatonic, Super Nordle, Sour Kush, Hindu Kush, Skunk Haze; Chromosomes (outlined) were stained with 2% acetocarmine and photographed under 400× magnification on a Zeiss Lab A1 microscope with an Axiocam 105 color camera;

FIG. 7 is a photograph of the rooting behavior of Cannabis tissue cultured in semi-solid media;

FIG. 8 is a photograph of explants showing the range of health scores (1-5) of Hindu Kush axillary bud explants at the time of transplant;

FIG. 9 is a graph showing DNA content measured by flow cytometry in cells from young leaves of Cannabis sativa strains Cannatonic (Panel A) and Skunk Haze (Panel B) measured on a Beckman Coulter Gallios flow cytometer;

FIG. 10 is a flow cytometry graph of genome size in cells from old leaves of the Cannabis sativa strain Hindu Kush measured on a Beckman Coulter Gallios flow cytometer, wherein the two peaks represent two groups of cells with different genome sizes (2C) and (4C);

FIG. 11 is a photograph of tetraploid Super Nordle plantlets recovering in culture;

FIG. 12 is a photograph of a underside of a leaf from Cannabis sativa onto which nail polish has been applied to several sections;

FIG. 13 is a picture showing the measurements for the guard cells of Cannabis sativa;

FIG. 14 is photographs of Stomata Morphology of nail polish impressions showing stomata on the abaxial surface of diploid (Panel A) and tetraploid (Panel B) Cannabis sativa Super Nordle mature fan leaves. Images were taken at 400× magnification on a Zeiss Lab.A1 microscope with an Axiocam 105 color camera. Samples taken from 3 month old mother plants. Scale bars: 25 microns;

FIG. 15 is graphs showing the comparison of growth parameters in diploid (orange, n=10) and tetraploid (blue, n=9) Super Nordle Cannabis sativa plants. Plants were transplanted 5 weeks after cloning. Plants were moved to the flowering room in week 4 and flowering lights were applied in week 5; Panel A: Plant height from soil to highest meristem; Panel B: Diameter of the stem one inch above the soil; Panel C: Width of the central leaflet in mature fan leaves; Panel D: Sum of the length of all lateral stems;

FIG. 16 is images showing leaf morphology for diploid (Panel A) and tetraploid (Panel B) fan leaves of Cannabis sativa Super Nordle collected in week 5 after transplanting in soil; Scale bar: 5 cm;

FIG. 17 is photographs showing trichome density on the adaxial surface of the 4th sugar leaf of diploid (Panels A-B) and tetraploid (Panels C-D) Super Nordle Cannabis sativa plants; Trichomes were denser in the tetraploid. Leaves were imaged on the 7th week of flowering. Scale bars: 1 mm;

FIG. 18 is photographs showing the inflorescence architecture of diploid and tetraploid plants, showing the cola of diploid (Panel A) and tetraploid (Panel B) Cannabis sativa Super Nordle during 8th week of flowering (week 12 after transplanting). Scale bars: 5 cm. (Panels C-D) Close-up morphology of buds from diploid and tetraploid, respectively. Scale bars: 1.5, 2.5 cm;

FIG. 19 is a graph showing the cannabinoid content in the dried buds and leaves of harvested Super Nordle Cannabis sativa plants as assessed by HPLC. B. Terpene profile. Upper and lower case letters indicate significant differences (p<0.05);

FIG. 20 is graphs showing the terpene profile in the dried buds and leaves of harvested Super Nordle Cannabis sativa plants, as assessed by gas chromatography; Panel A: Total terpene content (%); Panel B: Terpene content (mg g⁻¹ dry weight); and

FIG. 21 is photographs showing regeneration of tetraploid shoots for Cannabis sativa Super Nordle following oryzalin treatment of axillary bud explants; Panel A: deformed meristem structure at 5 weeks after oryzalin treatment; Panel B: Shoot initiation at 9 weeks, Scale bar: 5 mm; Panel C: Recovered shoot at 14 weeks, Scale bar, 15 mm; Panel D: Hardening plantlet at 19 weeks, Scale bar, 2 cm; Panel E: Mature tetraploid plant at 24 weeks, Scale bar, 8 cm.

### DETAILED DISCLOSURE OF EMBODIMENTS

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which variations and additions do not depart from the present technology. Hence, the following description is intended to illustrate some particular embodiments of the technology, and not to exhaustively specify all permutations, combinations and variations thereof.

As used herein, the singular form “a,” “an” and “the” include plural referents unless the context clearly dictates otherwise.

The recitation herein of numerical ranges by endpoints is intended to include all numbers subsumed within that range (e.g., a recitation of 1 to 5 includes 1, 1.5, 2, 2.75, 3, 3.80, 4, 4.32, and 5).

The term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

As used herein, the term “comprise” is used in its non-limiting sense to mean that items following the word are included, but items not specifically mentioned are not excluded.

As used herein, the term “strain” can be used interchangeably with “genotype” and refers to the DNA sequence of the genetic makeup of a cell, and therefore of a plant, which determines a specific characteristic (phenotype) of that plant. As used herein the term refers to different variants of a species of plant and is used interchangeably. Examples of strains or genotypes of Cannabis include, but are not limited to: Hindu Kush, Skunk Haze, Cannatonic, Super Nordle, Sour Kush, Acapulco Gold, Wonder Diesel, and Black Gold.

As used herein, the term “Cannabis” refers to the genus of flowering plants in the family Cannabaceae. Three species may be recognized as being part of the Cannabis genus, namely: Cannabis sativa, Cannabis indica, and Cannabis ruderalis. The expressions “Cannabis sativa” and “C. sativa” are used herein interchangeably.

As used herein, the term “diploid” refers to organisms or cells with two complete chromosome sets (2n), typical in the somatic cells of a plant. A Cannabis sativa diploid (2n) plant with a complete set of chromosomes has 20 chromosomes. As used herein, the term “polyploid” refers to a plant having more than the usual number (two) of chromosome sets, including three or more chromosome sets, four or more, five or more, etc. A true polyploid will have the extra chromosome sets in all cells, but ploidy can vary between tissues and is sometimes not passed on to the seeds. Polyploid can refer to organisms with three or more complete chromosome sets in all somatic cells. Polyploid can refer to organisms with three or more complete chromosome sets in one or more tissues. Polyploids can include, but are not limited to, triploids (3n), tetraploids (4n), hexaploids (6n), and octaploids (8n). As used herein, the expression “stable tetraploid plant” refers to a plant that retains its tetraploid number in some, most, or all tissues for a few months and/or through multiple generations.

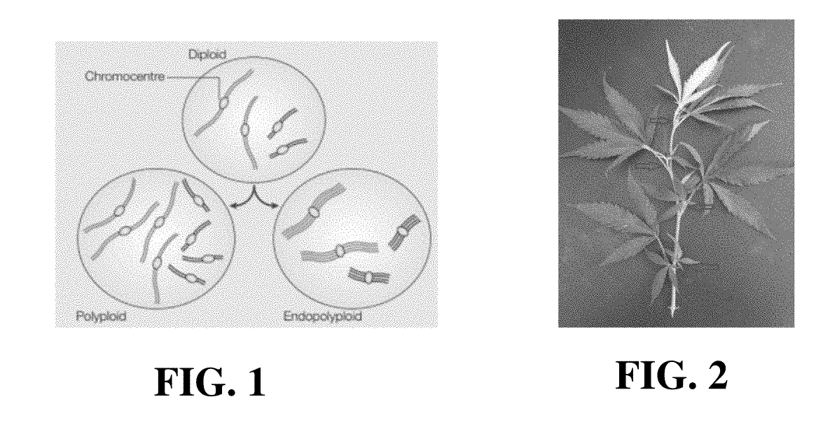

The term “endopolyploidy” as used herein refers to a natural doubling of the DNA content brought about by the process of endoreduplication in the plant cell which occurs when the cell undergoes a DNA replication without cell division, however, the number of chromosomes remains the same (FIG. 1). The term “aneuploid” as used herein refers to the situation when particular chromosomes are under or over-represented, but the entire chromosome set is not multiplied. For example, an aneuploid cell or plant may have 3 copies of chromosome numbers 1, 5, and 6, but only two copies of the other chromosomes.

As used herein, the term “mixoploid” plant refers to a plant having a mix of different ploidy cells within one tissue of the plant. For example, both diploid and tetraploid cells are present in the leaves. In some cases, the tissue or tissues are composed of some polyploid and some diploid cells.

As used herein, the expression “root tip squash” refers to a method whereby actively dividing cells from the root tips of a plant are isolated, stained, and mounted on a slide so that the chromosomes may be observed and/or counted under a microscope.

As used herein, the term “chlorosis” refers to the condition where leaves lose their green pigmentation, which can be caused by nutrient deficiency, lack of light, or disease.

As used herein, the term “vitrification/hyperhydricity” refers to a condition occurring in tissue cultured plants where too much water is taken into the plant, resulting in thin, weak leaves and poor stomata function.

Polyploidization is a powerful tool for improving desirable plant characteristics and is an effective method to induce variation. The method of polyploidization can result in a plant that has increased value for medicinal uses and a plant that is stable enough to use in the medical industry. Because of the allogamous nature of the fertilization of the species (the fertilization of a flower by pollen from another flower, especially one on a different plant), it is difficult to maintain the plant's potency and efficacy if grown from the seeds. Therefore, tissue culture is the most suitable way to maintain their genetic lines (although some of the plants used for medicinals are monoecious).

In one embodiment, the present technology relates to a method of inducing polyploidy in a Cannabis plant. In some implementations of this embodiment, the present technology relates to a method of inducing tetraploidy in a Cannabis plant. The present method has an advantage that the resulting plant is a clonal variant of mother plants.

Induction of polyploidy in Cannabis plants is obtained in the present technology by treating the Cannabis plant with an amount of a dinitroaniline compound. Examples of dinitroanilines that may be useful in the methods of the present technology include, but are not limited to: benfluralin, butralin, chlornidine, dinitramine, dipropalin, ethalfluralin, fluchloralin, isopropalin, methalpropalin, nitralin, oryzalin, pendimethalin, prodiamine, profluralin, and trifluralin or derivatives thereof. In some instances, the dinitroaniline compound is oryzalin or a derivative thereof. In some other instances, the dinitroaniline compound is trifluralin or a derivative thereof.

In some implementations, the method is performed on somatic plant tissue such as, but not limited to, auxiliary bud of the Cannabis plant. The treated somatic plant tissue is then allowed to grow in tissue culture and may then be planted in soil. As used herein the expression “treated” refers to a plant or a part thereof that has been treated with a dinitroaniline compound according to the embodiments of the present technology.

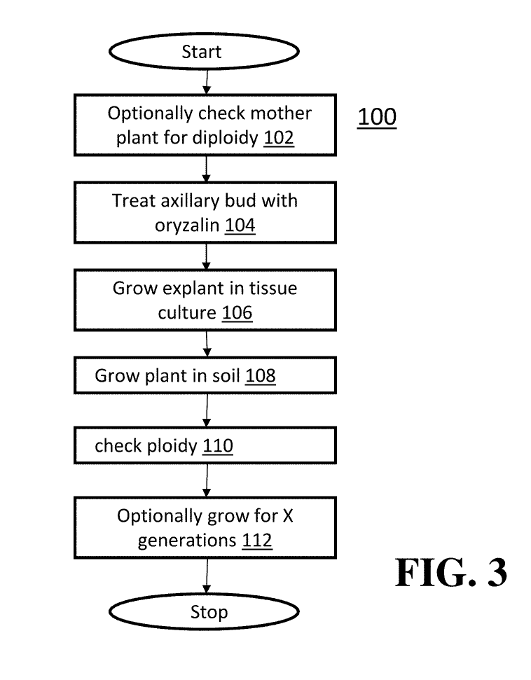

FIG. 3 outlines a method for inducing polyploidy (in particular tetraploidy) in Cannabis sativa according to one embodiment of the present technology, wherein oryzalin is used as the dinitroaniline compound. In this embodiment, the mother plants may first be optionally confirmed for diploidy (step 202) using methods known to those of skill in the art such as the root tip squash method (Example 1) and/or by flow cytometry. The axillary buds are then treated with oryzalin in an amount and for a time suitable to induce polyploidy (step 204). For instances, the auxiliary buds may be soaked in a solution of oryzalin at a concentration effective to induce polyploidy (step 204). The axillary buds of C. sativa can be excised using any method known in the art. The effective concentration of oryzalin may vary depending on the strain or genotype of C. sativa used. The method may include soaking the excised axillary buds of C. sativa in a concentration of oryzalin strong enough to induce polyploidy or more specifically, tetraploidy but not so strong that it induces toxicity in the axillary buds resulting in death of a high percentage of the resulting plants. The excised axillary buds of C. sativa are soaked in the composition of oryzalin for a time sufficient to induce polyploidy in the treated axillary bud. The time needed to induce polyploidy may vary depending on the strain or genotype being used. The treated bud is then transplanted and grown in tissue culture producing a plantlet (step 206) and is grown in tissue culture until it roots. The method of growth in tissue culture can include growth in a semi-solid media containing shoot elongation hormones. In some embodiments, the explant is kept in shoot elongation medium until it is ready to be transferred to medium containing a rooting hormone. In some embodiments, the explants are left in the shoot elongation medium until shoots form. The resulting plantlet is then transferred to soil, also called acclimatization or “hardening off” (step 208). The media from the tissue culture (including a gelling agent) can be gently broken up (with forceps or using any other method that does not result in damage to the roots) and the plantlet can be removed from the culture container.

In some embodiments, the plants are covered with a humidity dome and vented periodically to reduce the amount of humidity that builds up in the dome. In some embodiments, the plants are covered with a humidity dome for between about 1 and about 5 weeks, including but not limited to between about 1 week, about 1.5 weeks, about 2 weeks, about 2.5 weeks, about 3 weeks, about 3.5 weeks, about 4 weeks and about 4.5 weeks. In some embodiments, after between about 1 week and about 5 weeks in the humidity dome, the dome is removed and the plants are allowed to grow in a typical environment and the plant health is assessed with time.

In step 210, the plant, an explant, or a plantlet is tested for polyploidy using any methods know in the art. In some embodiments, one or more plant tissues is initially tested via flow cytometry and then the results are confirmed using the root tip squash method where the chromosomes in the root tip are imaged and counted. In some embodiments polyploidy is checked in the young leaves via flow cytometry. In some embodiments polyploidy is checked in both the young and older leaves via flow cytometry. In some embodiments, polyploidy is checked in the root tips via the root tip squash method. In some embodiments the plant is tested for mixoploidy.

The plant may be grown until it is flowering and tested for cannabinoids and other substances (step 212). At this point the plant may be allowed to grow for a few months to make sure it is a stable tetraploid. The plant may be allowed to grow for multiple generations to identify that it is a stable tetraploid.

In some other embodiments, method 200 may not have all of the above steps and/or may have other steps in addition to or instead of those listed above. The steps of method 200 may be performed in another order. Subsets of the steps listed above as part of method 200 may be used to form their own method.

According to some embodiments, the expression in “an amount sufficient to induce polyploidy” refers to a concentration of dinitoaniline compound which is effective to induce polyploidy in a Cannabis plant. In some implementations, the effective amount is between about 5 μM and about 200 μM, or between about 10 μM and about 200 μM, or between about 50 μM and about 200 μM, about 5 μM and about 100 μM, or between about 10 μM and about 100 μM, or between about 20 μM and about 150 μM, or between about 20 μM and about 60 μM, or between about 50 μM and about 150 μM, or between about 50 μM and about 100 μM; or is about 5 μM, or about 10 μM, or about 15 μM, or about 20 μM, or about 25 μM, or about 30 μM, or about 35 μM, or about 40 μM, or about 45 μM, or about 50 μM, or about 55 μM, or about 60 μM, or about 65 μM, or about 70 μM, or about 75 μM, or about 80 μM, or about 85 μM, or about 90 μM, or about 95 μM, or about 100 μM, or about 105 μM, or about 110 μM, or about 115 μM, or about 120 μM, or about 125 μM, or about 130 μM, or about 135 μM, or about 140 μM, or about 145 μM.

In some embodiments, the method of preparation of the dinitoaniline compound includes dissolving the dinitoaniline compound in a suitable solvent and then diluting the resulting composition with media to obtain a suitable or desired concentration of the dinitroaniline compound. In some embodiments, the media comprises sucrose and salts (for example, the media may comprise 30 g/L sucrose and 4.43 g/L MS basal salts). In such embodiments wherein the dinitoaniline compound is oryzalin, ethanol (e.g., 80%) is used because oryzalin is not soluble in water. The ethanol may also serve to disrupt cell membrane of the axillary bud to allow contact between the oryzalin and the chromosomes. The concentration of ethanol in the resulting oryzalin mixture may be less than about 0.5%, including but not limited to less than about 0.45%, about 0.4%, about 0.35%, about 0.3%, about 0.25%, about 0.2%, about 0.15%, about 0.1%, or about 0.5%. In some instances, the concentration of ethanol is less than about 0.25%. Optionally, before transplantation, the axillary bud is rinsed with sterile water with about 1 ml/L PPM™ (a plant preservative mixture). In other instances, the concentration of PPM™ is between about 0.5 and about 2 ml/L PPM™ including about 0.6, about 0.7, about 0.8, about 0.9, about 1.0, about 1.1, about 1.2, about 1.3, about 1.4, about 1.5, about 1.6, about 1.7, about 1.8, or about 1.9 ml/L PPM™.

According to some embodiments, the expression in “a time sufficient to induce polyploidy” refers to a period of time during which the Cannabis plant is treated with the dinitroaniline compound that is sufficient to induce polyploidy in the Cannabis plant. In some implementations, the time sufficient to induce polyploidy is between about 24 hours and about 48 hours, or between about 12 hours and about 48 hours, or is about 13 hours, about 14 hours, about 15 hours, about 16 hours, about 17 hours, about 18 hours, hours 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, hours 36 hours, about 37 hours, about 38 hours, about 39 hours, about 40 hours, about 41 hours, about 42 hours, about 43 hours, about 44 hours, about 45 hours, about 46 hours, about 47 hours, or about 48 hours.

In some embodiments, shoot elongation medium is 4.44 g/L Murashige & Skoog (MS) basal media with vitamins. In some embodiments, the shoot elongation medium includes naphthaleneacetic acid (NAA). In some embodiments, the shoot elongation medium includes kinetin (KIN). In some embodiments, the NAA is included at a concentration of between about 0.05 to about 0.5 mg/L, including about 0.1 mg/L to about 0.5 mg/L, including but not limited to about, 0.2, 0.3, and 0.4 mg/L. In some embodiments, the kinetin is used at a concentration of about 0.2 to about 2.0 mg/L. In some embodiments, the KIN is included at a concentration of about 0.4 mg/L to about 1 mg/L, including but not limited to about, 0.5, 0.6, 0.7, 0.8, and 0.9 mg/L.

In some embodiments, the explant is kept in the shoot elongation medium until it is ready to be transferred to medium containing a rooting hormone. In some embodiments, the explants are left in the shoot elongation medium until shoots form. In some embodiments, the explant (treated axillary bud) is grown in a semi-solid media containing a gelling agent such as Gelzan™ or Agar. In some embodiments the Gelzan™ is included at a concentration of about 4 mg/L. In some embodiments, charcoal is added to the semi-solid media at a concentration of from about 0.1 mg/L to about 1 mg/L, including 0.5 mg/L. In some embodiments, the amount of time the explants are left in the shoot elongation medium is between about 1 week and about 16 weeks, or is about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks or about 15 weeks total. In some embodiments, the time required for rooting and obtaining shoots is between about 4 weeks and about 8 weeks. In some embodiments, the explants start rooting in the elongation media and do not require rooting media.

In some embodiments, the explant is transferred to a media containing a rooting hormone. In some embodiments, the rooting hormone comprises indole-3-butyric acid (IBA). In some instances, the IBA is used at a concentration of between about 0.5 mg/L and about 2 mg/L, or at a concentration of about 0.6 mg/L, about 0.7 mg/L, about 0.8 mg/L, about 0.9 mg/L, about 1.0 mg/L, about 1.1 mg/L, about 1.2 mg/L, about 1.3 mg/L, about 1.4 mg/L, about 1.5 mg/L, about 1.6 mg/L, about 1.7 mg/L, about 1.8 mg/L, or about 1.9 mg/L. In some instances, the time required for rooting is between about 1 week and about 16 weeks, or is about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks or about 15 weeks. In some embodiments, the time required for rooting is between about 4 weeks and about 8 weeks.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1

### Chromosome Counting Method

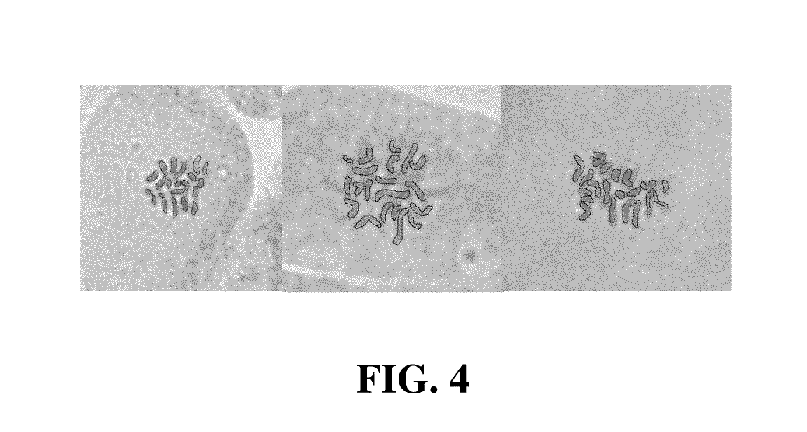

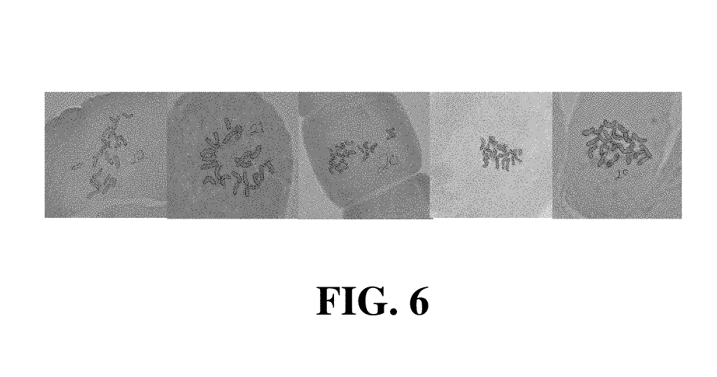

Chromosomes were observed in C. sativa using the root tip squash method. Briefly, root tips were harvested, and fixed in 3:1 ethanol:acetic acid for 24 hours. The roots were then hydrolyzed in 1M HCl for 2 minutes at 60° C. and rinsed with ice cold water three times. Roots were stained in 2% acetocarmine for 2 hours at 60° C. To prepare the slides, a few millimeters of the root tip was removed from each root, macerated with a scalpel, and crushed with a flat metal surface. A coverslip was applied and tapped gently to spread out the cells. The slide was heated gently on a hot plate and squashed between filter papers. FIG. 4 shows chromosomes in the root tip cells of Hindu Kush C. sativa. The individual chromosomes have been outlined for clarity. Cells were stained with 2% acetocarmine and photographed under 1000× magnification on a Zeiss Lab A1 microscope with an Axiocam 105 color cameral. In the photograph, the Hindu Kush is diploid, showing 20 chromosomes. The cells were identified under low magnification and then photographed under the 100× objective with oil. Further experiments determined that the process could be used for other genotypes, including those used in the following examples (see also FIG. 6). In addition, before use, the mother strains were tested to confirm they were diploid.

### Example 2

### Axillary Bud Culture Methods

Axillary bud regeneration methods were established as provided in the following example. Growth in semi-solid media containing various shoot elongation hormones was tested to identify the best conditions for tissue culture of axillary bud from C. sativa. In Table 1, Skunk Haze C. sativa axillary buds were grown in semi-solid media containing 30 g/L sucrose, 4.43 g/L Murashige & Skoog basal nutrient media, and 8 g/L agar. Three hormone combinations were tested: 0.5 mg/L metatopolin (mT) (Lata et al. 2016), 0.5 mg/L of thidiazuron (TDZ) and 7 mg/L of gibberellic acid (GA3) (Lata et al. 2009), and 0.3 mg/L of 1-Naphthaleneacetic acid (NAA) and 0.4 mg/L of kinetic (KIN) (determined to be effective in previous trials). Sterilized portions of the axillary bud (explants) were implanted in each of the three types of media and the explants were grown for 4 weeks at approximately 50 mol/m²/sec light on a 16 h day cycle. The height of the explants was measured weekly to assess the growth and the health of the explants was scored. The health scoring system used in the Examples herein was as follows: 0=explant is dead; 1=explant is very small, very deformed, and/or showing a lot of chlorosis and necrosis; 2=Poor growth, mild necrosis or severe chlorosis, deformed; 3=moderate growth, some chlorosis or deformity; 4=mild deformity or chlorosis; and 5=excellent health, green. Initial results in Table 1 indicated that 2 μM metatopolin (Lata et al. 2016) was superior to TDZ and GA3 (Lata et al. 2009), or NAA and Kinetin for shoot elongation, but the plants eventually developed deformed leaves which were very thin and curled. Later tests using NAA, KIN, and GA3 resulted in very unhealthy plants after 2 weeks which were discarded.

TABLE 1
Growth and health of Skunk Haze C. sativa
axillary buds over 4 weeks in semi-solid MS
media with various hormone combinations (n = 9)

| Axillary bud regeneration | | shoot length (cm) | | health score | |
|---|---|---|---|---|---|
| test #1 | | average | SE | average | SE |
| TDZ + GA3 | week 2 | 1.50 | 0.23 | 2.22 | 0.22 |
| | week 3 | 1.69 | 0.23 | 2.33 | 0.17 |
| | week 4 | 1.82 | 0.25 | 2.78 | 0.22 |
| NAA + KIN | week 2 | 1.28 | 0.18 | 4.56 | 0.18 |
| | week 3 | 1.69 | 0.17 | 4.11 | 0.20 |
| | week 4 | 2.38 | 0.20 | 3.22 | 0.28 |
| mT | week 2 | 1.74 | 0.14 | 4.67 | 0.17 |
| | week 3 | 2.24 | 0.16 | 4.00 | 0.17 |
| | week 4 | 2.94 | 0.26 | 4.22 | 0.15 |

In Table 1, SE refers to the standard error, a measure of variability within the treatment. n=9 refers to the number of plants. Further trials with the God Bud 2 variety of C. sativa indicated that explants grew very well in hormone free media. The explants were generally healthy, though they were fairly chlorotic after 4 weeks. The explants grown in hormone free media did not show the deformed phenotype which was present in the metatopolin trials. Because metatopolin resulted in the best shoot elongation for Skunk Haze, a new trial was initiated with half the original concentration of metatopolin (1 μM instead of 2 μM), to identify a method that would result in less deformed and healthier plants (which did not become chlorotic). While metatopolin typically acts as a rooting hormone (Lata et al. 2016), none of the explants still on metatopolin rooted after 4 weeks (though they still looked healthy and did not have the large callus mass around the base). When some of these explants were moved to standard rooting media with indole-3-butyric acid (IBA) as the rooting hormone (½ strength MS with 1 mg/L IBA), several of the metatopolin (mT) and NK explants developed roots. Both NK and mT explants grew roots, but the roots seemed to be growing upward or sideways out of the callus mass at the base, and not down into the media. During the most recent subculture, the callus masses at the base of the shoots were pushed down into the media to encourage the roots to grow down into the media. Following the transfer to fresh rooting media, the roots did not continue to grow. Additional rooting trials involved using full MS media (Movahedi et al. 2015, Slusarkiewicz-Jarzina et al. 2005), a lower concentration of IBA: 0.5 mg/L (Chaohua et al. 2016, Movahedi et al. 2015), and comparing 8 g/L agar to 4 g/L Gelzan™. After one week the plants were growing well, showing some chlorosis and a mild twisted leaf deformity. These experiments showed that Gelzan™ was more advantageous for a number of reasons. The trials were conducted as before except that vented caps were used to reduce hyperhydricity. The vented caps were effective, since only one explant (out of 10) had mild hyperhydricity symptoms. Because the results using shoot elongation media did not identify a clear advantage and the metatopolin tended to result in more deformation, future trials used media with IBA (0.5 mg/L or 1 mg/L) as a rooting hormone. However, tests did show that Gelzan™ was more advantageous over agar because it produced a softer media less inhibitory to root growth, and was also much clearer than agar, so new roots were easier to spot. However, this was not an issue when charcoal was used in the media because the media became opaque. In addition, as shown in Table 2 below, while both types of gelling agents were effective on the five plants of God Bud 2 tested, roots developed faster and more consistently using Gelzan™ based on the rooting speed (d=days). SE refers to the standard error, a measure of variability within the treatment. Thus, Table 2 showed that using Gelzan, the plants rooted more quickly and more uniformly based on the standard error, making it more effective for efficient propagation and production.

TABLE 2
Effect of different gelling agents on rooting
speed of “God Bud 2” C. sativa

Rooting speed (d)

| Gelling agent | average | SE |
|---|---|---|
| Agar (8 g/L) | 27.6 | 4.83 |
| Gelzan ™ (4 g/L) | 19 | 0.63 |

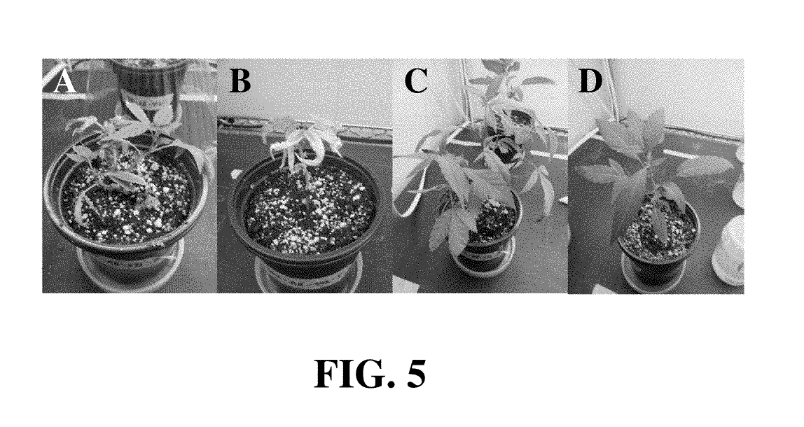

Previous trials with two plants which successfully rooted in media showed that plants could effectively be moved from culture to soil without too much difficulty (“hardening off”). Plants that rooted were moved from culture to soil as follows: media was gently broken up with forceps and the plant was removed from the culture container. Media was rinsed off of the roots with distilled water and necrotic or senescing tissue was removed. The plantlets were placed in small pots with soil and covered with a humidity dome for 1 week, and incubated on a culture shelf. Initially the explants looked fairly unhealthy due to the extended time in culture, but they recovered well. FIG. 5 provides images showing an example of movement from culture to soil. The left two images were immediately after transplant, while the right two images were after 1 month in soil. Even after 1 month in soil, the plants looked a bit deformed—most notably, there were often fewer leaflets than expected for the genotype and stems developed a red color which was likely indicative of stress from nutrient deficiency. There was concern that the addition of PPM™, a plant preservative, might be stunting the growth of the plants. Therefore, a trial was run with Hindu Kush using the standardized shoot elongation media (30 g/L sucrose, 4.43 g/L MS, g/L charcoal, 8.0 g/L agar) and either 0.5 or 1.0 mL/L PPM™. The growth and health of the plants was assessed over 4 weeks, as well as keeping track of the contamination rate. There was no significant difference in any of the parameters as shown by Table 3 below:

TABLE 3
Plant growth assessment with PPM ™ treatment (n = 10)

| PPM ™ | | height | | health | | # |
|---|---|---|---|---|---|---|
| treatment | week | ave | SE | ave | SE | contaminations |
| 1.0 ml/L | 1.00 | 0.71 | 0.09 | 5.00 | 0.00 | 3.00 |
| | 2.00 | 1.27 | 0.20 | 4.33 | 0.16 | |
| | 3.00 | 1.50 | 0.23 | 4.11 | 0.19 | |
| | 4.00 | 1.66 | 0.25 | 4.11 | 0.25 | |
| 0.5 ml/L | 1.00 | 0.69 | 0.06 | 5.00 | 0.00 | 3.00 |
| | 2.00 | 1.20 | 0.14 | 4.22 | 0.26 | |
| | 3.00 | 1.62 | 0.27 | 3.78 | 0.26 | |
| | 4.00 | 1.72 | 0.30 | 3.78 | 0.26 | |

The results in Table 3 showed that the addition of 1 ml/L PPM™ did not affect the growth or health of the plant.

### Example 3

### Trial Using Oryzalin to Clarify Procedure

A trial was conducted using oryzalin to clarify the procedure to produce polyploid C. sativa using the methods of culture and chromosome counting in Examples 1 and 2. The media used in this trial was an early media. A different media was developed for later trials. The oryzalin treatment of axillary buds was as follows: Axillary buds at early stages of development were chosen (e.g., no large leaves emerged). All explants were taken from a single mother for consistency. The explants placed in water in a 4° C. refrigerator overnight (cold treatment was used to reduce oryzalin toxicity and to slow down mitosis so the oryzalin could take effect) and sterilized. Later experiments skipped the refrigeration step and used fresh samples instead. Axillary buds were immersed in liquid MS media (supplemented with 30 g/L sucrose, no hormones) with 2.5 μM or 5.0 μM oryzalin for 24 and 48 hours prior to introduction onto shoot growth media. Explants were sterilized and then swirled in 50 mL falcon tubes with the oryzalin media on a shake table (110 RPM). Following the oryzalin treatment, the explants were rinsed three times in sterile distilled water to remove remaining oryzalin, and plated on shoot elongation media. Explants were cultured as discussed in Example 2. Using the root tip squash procedure, all of the strains were identified as diploid. Previous experiments showed that all of the Cannabis chromosomes were metacentric (1 set was sub metacentric) and about the same size (the X chromosome is slightly larger) (Divashuk et al. 2014), which was helpful in identifying individual chromosomes. FIG. 6 shows the prophase chromosomes in the root tip cells of the following C. sativa genotypes from left to right: Cannatonic, Super Nordle, Sour Kush, Hindu Kush, and Skunk Haze. Chromosomes were stained with 2% acetocarmine and photographed under 400× magnification on a Zeiss Lab A1 microscope with an Axiocam 105 color camera. For counting, the chromosomes were outlined for clarity. The treated axillary buds were cultured as follows: Murishige & Skoog media (MS media), 8.0 g/L agar for the elongation stage. These trials were done with NK charcoal media and were later moved to the following rooting media: MS, 0.3 mg/L charcoal, 1.0 mg/L IBA, agar. The first oryzalin treated plants rooted.

Trials for rooting effectiveness: Using the God Bud 2 axillary buds which had been grown for the hormone free shoot elongation test (see Example 2), a trial was run to compare rooting effectiveness on MS media with either 8.0 mg/L agar or 4.0 mg/L Gelzan™. In this experiment, all of the explants rooted within 40 days. Therefore, it is likely that the hormone free incubation during shoot elongation encouraged rooting. Comparing the two gelling agents, the Gelzan™ explants rooted about 10 days sooner (an average of 19 days, rather than 28 days), and rooted more consistently than the agar treatment (agar explants rooted between 13-39 days). Therefore, for future trials Gelzan™ was used for the rooting phase. Using the optimal basal media and gelling agents identified above (MS media with 4.0 mg/L Gelzan™), experiments were performed to compare hormone combinations, and to see whether a period of hormone-free incubation was beneficial for rooting. 0.5 g/L of charcoal was added to both the elongation and rooting media for this experiment. The use of 0.3 mg/L NAA and 0.4 mg/L KIN for axillary bud elongation, was compared to that using 2 μM metatopolin (˜0.5 mg/L). Each treatment began with 10 explants each, then after two weeks, each treatment was divided in half, with 5 transferred to fresh NK or mT media, and 5 of each transferred to hormone-free media for the last two weeks of shoot elongation. As shown in Table 4, after two weeks on the charcoal-containing media, explants having both treatments (the number of plants tested “n” was 5, SE refers to the standard error, a measure of variability within the treatment) were still generally healthy, with chlorosis developing around the leaf tips and margins, a bit more in the mT treatments. The metatopolin plants were also developing slightly deformed leaves. Both metatopolin and NK explants were growing well. At the end of the 4^{th} week, growth was faster on the media with hormones, but, hormone free treatments had better health scores than their hormone counterparts. Both hormone treatments resulted in narrow, curled young leaves, which were more pronounced on mT. Based on these results, NK media for two weeks followed by two weeks in hormone free media was the best for both growth and health of the plants.

TABLE 4
Development of Hindu Kush axillary buds over 4 weeks in various
hormone treatments (n = 5)

| Treatment | | | Health | | Growth (mm) | |
|---|---|---|---|---|---|---|
| week 1-2 | week 3-4 | week | ave | SE | ave | SE |
| mT | mT | 1 | 5.00 | 0.00 | 9.19 | 2.07 |
| | | 2 | 4.20 | 0.20 | 7.26 | 0.85 |
| | | 3 | 3.80 | 0.20 | 0.76 | 0.96 |
| | | 4 | 3.40 | 0.24 | 3.33 | 0.91 |
| | HF | 1 | 4.80 | 0.20 | 6.12 | 1.54 |
| | | 2 | 4.20 | 0.37 | 7.04 | 1.09 |
| | | 3 | 3.80 | 0.49 | 2.67 | 0.79 |
| | | 4 | 3.60 | 0.40 | 1.90 | 0.85 |
| NK | NK | 1 | 4.80 | 0.20 | 9.13 | 1.84 |
| | | 2 | 4.20 | 0.37 | 5.10 | 0.41 |
| | | 3 | 3.80 | 0.49 | 1.34 | 1.31 |
| | | 4 | 4.00 | 0.00 | 2.66 | 1.06 |
| NK | HF | 1 | 5.00 | 0.00 | 9.73 | 2.10 |
| | | 2 | 4.60 | 0.24 | 6.22 | 1.02 |
| | | 3 | 4.60 | 0.24 | 2.51 | 0.90 |
| | | 4 | 4.20 | 0.20 | 2.54 | 1.17 |

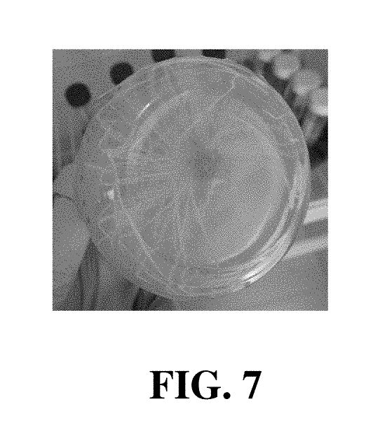

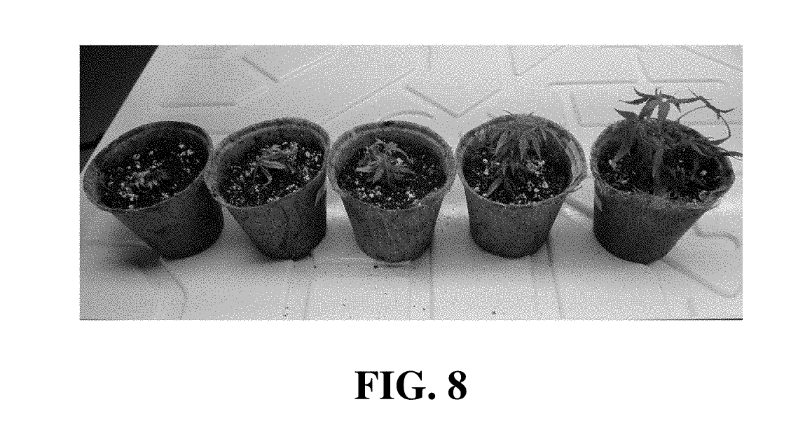

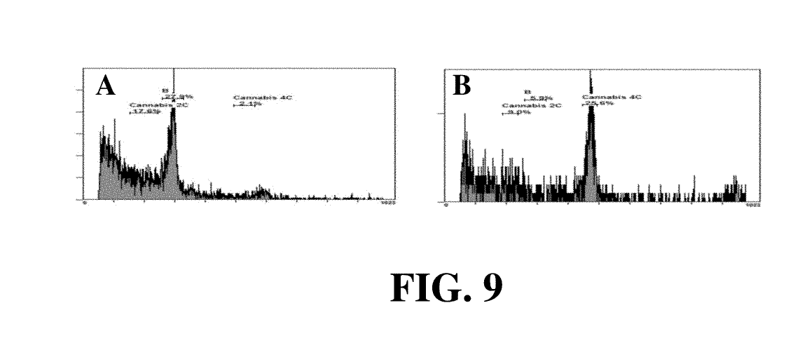

As shown in Table 4, to assess rooting, the Hindu Kush explants were checked daily for the development of roots. FIG. 7 shows an example of a plant rooting in the tissue culture process. The plant was rooted in semisolid media (agar in this case). The NK treatment was the least successful, because only one of the three plants rooted on the last day. The hormone free (HF) treatment did not seem to matter for the metatopolin treated plants. Table 4 provides the results of rooting, including the percent of the plants that rooted, average days for rooting, and average health at the end of the trial (using the health rating system in Example 2). SE refers to the standard error, a measure of variability within the treatment. All of the plants that survived the treatment were used to assess rooting. Therefore n=5, 4, 3, and 5 relates to the number of plants that survived and were used for analysis for each treatment from top to bottom. In other words, 5 metatopolin treated plants were assessed for rooting, 4 metatopolin+HF were assessed, 3, NAA/KIN were assessed and 5 NAA/KIN+HF were assessed. Plant health was comparable between all treatments at the end of the rooting period. The plants were showing a lot of chlorosis, leaf tip burn, and deformed leaves (especially the young ones). The NK/HF plants also seemed to have some stem tip dieback. Overall, the metatopolin treatment was somewhat more effective when considering both shoot growth and rooting. The conclusions were that if the NAA/KIN hormone combination was used for shooting, it should include 2 weeks of hormone free incubation to improve rooting success, but this was not necessary for metatopolin treatment. All of the rooted plants were transplanted into soil for a larger hardening off trial, using the same methods outlined in Example 2. In general, the plants all grew well while under the humidity domes. Once the domes came off, a couple of the smaller plants died. There was a significant correlation between the initial health and rooting score and the health of the explants after two weeks. By the end of the hardening process, most plants were very healthy. The few exceptions were the plants which did not have well developed roots when they were transplanted. This suggested that, after an explant roots, leaving it in the rooting media for an additional week (or several weeks depending on the speed of root development) allows it to better develop its roots and the shoots to recover before transplanting. FIG. 8 shows the range of health scores from 1 to 5 (from left to right in FIG. 7) for Hindu Kush axillary bud explants at the time of transplant. The health scores for Tables 4 and 5 are all based on the health rating system provided in Example 2: 0: explant is dead, 1: explant is very small, very deformed, and/or showing a lot of chlorosis and necrosis, 2: Poor growth, mild necrosis or severe chlorosis, deformed, 3: moderate growth, some chlorosis or deformity, 4: mild deformity or chlorosis, and 5: excellent health, green.

TABLE 5
Rooting in axillary bud explants of Hindu Kush
after various shoot elongation treatments

| | | average | Average |
|---|---|---|---|
| | percent | days to | health at end |
| Treatment | rooted | rooting | of trial |
| Metatopolin | 100 | 23.6 | 3.4 |
| Metatopolin - HF | 75 | 23.3 | 3.75 |
| NAA/KIN | 33 | 34 | 3.4 |
| NAA/KIN - HF | 100 | 26.6 | 3.33 |

### Example 4

### Oryzalin Treatment (First Trial)

In Examples 1-3 the axillary bud regeneration process was optimized for untreated buds and, using this information, the trial using oryzalin treatment to induce polyploidy was started. However, the first trial started before the final rooting data in Example 3, so the NK-HF treatment was used. The buds were treated with 2.5 or 5.0 μM oryzalin for either 24 or 46 hours. A negative control with no oryzalin had the same concentration of ethanol (oryzalin carrier) as that added to the 5.0 μM oryzalin treatment and was incubated for 46 hours to represent the strongest possible treatment. While the ethanol controls grew the fastest, they were particularly deformed and unhealthy looking (very long internodes). Conversely, as shown in Table 6, the oryzalin treated plants grew well (the number of plants tested “n” was 6 but some were lost due to contamination). SE refers to the standard error, a measure of variability within the treatment. There was only one explant which died after treatment in the group treated with 5 μM oryzalin for 24 hours. The explants treated with 2.5 μM oryzalin grew better than those treated with 5 μM for both 24 and 46 hours. However, the explants receiving the longer oryzalin treatment (5 μM for 46 hours) were the healthiest at the end of the shoot elongation period. Therefore, a high concentration of oryzalin slowed growth without significantly affecting plant health.

TABLE 6
Growth and health of Cannatonic axillary bud explants
following the indicated treatment

| oryzalin treatment | | | growth over | | | |
|---|---|---|---|---|---|---|
| length | concentration | | the week (mm) | | health | |
| (h) | (μM) | week | average | SE | average | SE |
| 24 | 2.5 | 1 | 11.68 | 1.69 | 5.00 | 0.00 |
| | | 2 | 8.87 | 1.82 | 5.00 | 0.00 |
| | | 3 | 3.50 | 1.24 | 4.83 | 0.17 |
| | | 4 | 2.71 | 0.61 | 4.17 | 0.17 |
| | 5 | 1 | 8.36 | 0.79 | 4.50 | 0.50 |
| | | 2 | 5.82 | 1.17 | 4.33 | 0.67 |
| | | 3 | 2.28 | 0.33 | 4.50 | 0.29 |
| | | 4 | 1.63 | 0.85 | 3.75 | 0.48 |
| 46 | 2.5 | 1 | 9.45 | 0.73 | 5.00 | 0.00 |
| | | 2 | 9.39 | 1.34 | 4.80 | 0.21 |
| | | 3 | 3.10 | 1.91 | 4.50 | 0.34 |
| | | 4 | 4.40 | 1.41 | 4.17 | 0.31 |
| | 5 | 1 | 8.33 | 1.24 | 4.67 | 0.21 |
| | | 2 | 5.08 | 0.31 | 4.50 | 0.34 |
| | | 3 | 2.55 | 1.27 | 4.80 | 0.20 |
| | | 4 | 1.66 | 1.71 | 4.60 | 0.24 |
| | 0 | 1 | 10.37 | 1.09 | 4.83 | 0.17 |
| | | 2 | 12.78 | 1.11 | 3.50 | 0.22 |
| | | 3 | 9.23 | 3.98 | 2.83 | 0.17 |
| | | 4 | −0.56 | 3.53 | 2.80 | 0.20 |

All of the explants were moved to rooting media and rooted well. The controls recovered once they were on the rooting media. The explants receiving the longer oryzalin treatment (5 μM for 46 hours) continued to be the healthiest and grew the best. Once a plant roots, it tends to recover and goes through a growth spurt, so the growth rate increased for plants with some treatments where several plants have rooted. Some of the plants had a strangely deformed meristem, which looked like a clump of tissue.

The first oryzalin trial was analyzed by flow cytometry of young leaves and measured on a Beckman Coulter Gallios flow cytometer combined with root tip analysis (see Example 1). The process allowed for optimization of the process/settings for running Cannabis samples through a flow cytometer. Making the cell suspension from the young leaves with LB01 buffer was most effective, and the machine parameters used were 465V for 120 s on the high setting (though for some poorer quality samples this was changed to 360 s on the medium setting). All five mother strains were confirmed to be diploid (FIG. 9A), consistent with the findings of the root tip squashes. Although most of the plants treated in the first oryzalin trial rooted and survived transplant, none of the plants from this trial were tetraploid.

TABLE 7
Preparation of LB01 buffer for flow cytometry (pH 8.0)

| Ingredient | Concentration (mM) | ~Mass in 500 mL (mg) |
|---|---|---|
| Tris | 15 | 908 |
| Na₂EDTA | 2 | 372 |
| Spermine-4HCl | 0.5 | 87 |
| KCl | 80 | 0.5 |
| NaCl | 20 | 0.2 |
| Triton X-100 | 0.1% v/v | 500 □L |

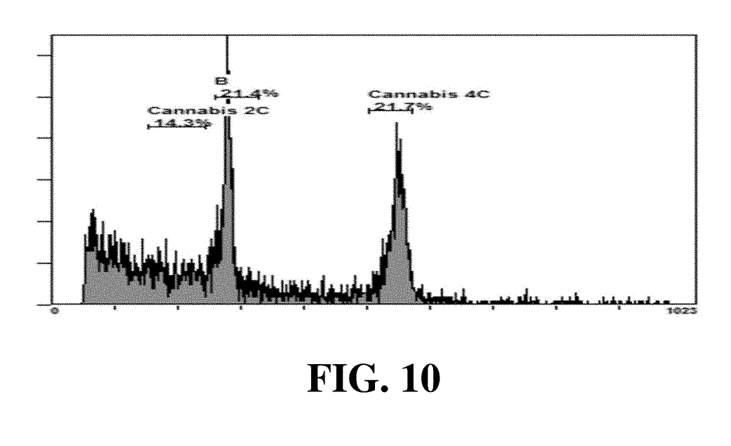

However, the analysis of the mother lines via flow cytometry identified some other results. The reported average size of a Cannabis genome is around 1.83 pg (Faux et al. 2013), which appears to match with most of the results. The genome sizes for Cannatonic (CAN), Super Nordle (NOS), Hindu Kush (HKU), and Sour Kush (SOK) were 1.96, 1.94, 1.92, and 1.94 pg, respectively. The discrepancy with earlier work was likely due to the fact that a radish standard was used instead of Maize. However, Skunk Haze (SHA) had a genome size of 3.02 pg. This size difference can be seen in FIG. 9 which shows the DNA content in cells from young leaves of C. sativa strains Cannatonic (a) and Skunk Haze (b). When comparing the flow cytometer histograms from Cannatonic (FIG. 9A) and Skunk Haze (SHA) (FIG. 9B) there is a clear difference. The results showing a genome size of 3.02 pg suggests that the genome size is not fully doubled, so, it is possible that this strain is aneuploid—having only a few doubled chromosomes. SHA was also less successful in the root tip squash method in Example 1 than the other genotypes, and the chromosomes were harder to count. Further experiments done in triplicate showed that the averages were: SOK: 1.96 HKU: 1.94 NOS: 1.97 SHA: 2.96 CAN: 1.97 (pg). Another phenomenon analyzed in the mother lines via flow cytometry was endopolyploidy—a natural doubling of the DNA content in older cells which occurs in about 90% of flowering plants, and can serve several functions within the plant (Scholes & Paige 2014). Endopolyploidy is not true polyploidy, since the DNA content in the meristem cells and young tissues remains unchanged, so genome size was analyzed in old leaves. While Super Nordle (NOS), Skunk Haze (SHA), Sour Kush (SOK), and Cannatonic (CAN) did not show any differences between their old and young leaf genome sizes, Hindu Kush had doubled DNA content in about 58% of the cells in the old leaf, indicating endopolyploidy (FIG. 9). FIG. 10 shows the genome size in cells from old leaves of the C. sativa strain Hindu Kush. The two peaks represent two groups of cells with different genome sizes (2C and 4C).

### Example 5

### Oryzalin Treatment (Second Trial)

A second trial was started for treating C. sativa with higher oryzalin concentrations. To find the range where oryzalin becomes lethal, this trial tested up to the higher range of effective concentrations: 50, 100, and 150 μM, in addition to the control on both Hindu Kush and Super Nordle. The method was as discussed in Example 4, but the oryzalin treatment concentrations were greater. The results in Table 8 showed that, although most of the explants looked relatively healthy immediately after treatment, there was a high rate of explant death, especially at the highest concentrations of oryzalin, and especially for Hindu Kush (HKU). In the Table, SE refers to the standard error, a measure of variability within the treatment. A number of the Super Nordle (NOS) plants were lost to contamination. During the 4 weeks of incubation on shoot elongation media, the health of the surviving plants also deteriorated, and most were not showing new growth (Table 7), and had the globular meristem deformity. It was not clear how effectively these explants would root in their present state, so they were kept on shoot elongation media a bit longer. The results showed that they recovered, although the time for recovery was variable. It was at least a month before they began showing new shoots, and 2-4 months before an individual explant had enough leaf material to take a sample for flow cytometry, then usually another few weeks before they were moved to rooting. The few that rooted on the shoot elongation media for up to 3 weeks until the roots developed enough were left for a couple of weeks and then potted in soil for hardening off. However, if they rooted on elongation media, they were left to develop for a few weeks before transplanting.

TABLE 8
Growth and health of oryzalin treated axillary buds over
the first three weeks of growth

| | Oryzalin | | growth over | | | |
|---|---|---|---|---|---|---|
| | Treatment | | the week (mm) | | health | |
| Genotype | (μM) | week | average | SE | average | SE |
| Hindu | 0 | 1 | 0.18 | 0.05 | 3.56 | 0.24 |
| Kush | | 2 | 0.14 | 0.05 | 3.25 | 0.31 |
| | | 3 | 0.06 | 0.05 | 2.75 | 0.16 |
| | 50 | 1 | −0.01 | 0.03 | 2.44 | 0.41 |
| | | 2 | 0.12 | 0.06 | 2.86 | 0.26 |
| | | 3 | −0.02 | 0.05 | 2.71 | 0.29 |
| | 100 | 1 | 0.12 | 0.06 | 2.00 | 0.24 |
| | | 2 | 0.01 | 0.05 | 1.60 | 0.24 |
| | | 3 | −0.11 | 0.10 | 1.60 | 0.24 |
| | 150 | 1 | −0.07 | 0.05 | 2.00 | 0.00 |
| | | 2 | 0.12 | 0.04 | 2.00 | 0.00 |
| | | 3 | −0.11 | 0.02 | 1.67 | 0.33 |
| Super | 0 | 1 | 0.23 | 0.07 | 4.83 | 0.18 |
| Nordle | | 2 | 0.94 | 0.32 | 4.00 | 0.00 |
| | | 3 | 0.31 | 0.22 | 4.00 | 0.41 |
| | 50 | 1 | 0.08 | 0.03 | 3.13 | 0.48 |
| | | 2 | 0.13 | 0.05 | 2.86 | 0.51 |
| | | 3 | 0.25 | 0.12 | 2.33 | 0.42 |
| | 100 | 1 | 0.17 | 0.03 | 3.29 | 0.36 |
| | | 2 | 0.12 | 0.05 | 2.40 | 0.51 |
| | | 3 | −0.06 | 0.07 | 2.33 | 0.88 |
| | 150 | 1 | 0.10 | 0.09 | 2.83 | 0.31 |
| | | 2 | 0.13 | 0.12 | 2.50 | 0.50 |
| | | 3 | −0.05 | 0.15 | 2.25 | 0.25 |

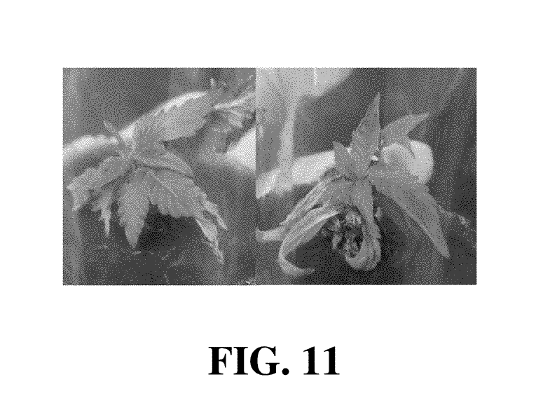

Some of the plants seemed to be recovering following the switch back to NK media. Some of the plants that had developed a highly deformed meristem were regenerating shoots and appeared to be developing small lateral shoots. These shoots were grown for a longer period of time and rooted. The results are shown in Table 9 for two C. sativa genotypes: Hindu Kush and Super Nordle. Of the 10 plants tested, 20-50% survived and of those most were mixoploid. However, one tetraploid Super Nordle plant survived. The results in Table 9 showed that several of the surviving plants from the high concentration oryzalin experiment were successfully transformed. These plants grew from small shoots that emerged from the very deformed meristems. Some of the plants from this experiment had not recovered enough to sample leaves at the time of testing, but will soon be ready to analyze using the flow cytometer. All of the control plants were diploid. One of the Super Nordle tetraploids rooted and grew well, though the leaves continued to look a little deformed (it should be noted that this is not unusual for a plant that has been in culture for that long). FIG. 11 provides a photograph of the tetraploid Super Nordle plantlets recovering in culture. These plants were monitored for abnormal growth and development.

TABLE 9
Results of the high oryzalin concentration trial

| | Oryzalin | Survival | Number of | | Number of |
|---|---|---|---|---|---|
| | dose | rate | 2n/4n | Number of | 4n/8n |
| Genotype | (μM) | (%) | mixes | tetraploids | mixes |
| Hindu | 50 | 60 | 3 | 0 | 0 |
| Kush | 100 | 0 | 0 | 0 | 0 |
| | 150 | 10 | 0 | 0 | 0 |
| Super | 50 | 30 | 0 | 1 | 1 |
| Nordle | 100 | 10 | 0 | 1 | 0 |
| | 150 | 10 | 0 | 0 | 0 |

After using the standardized shoot elongation media, an experiment was performed to confirm that it worked for all of the strains. The media used was 0.1 mg/L NAA, 0.4 mg/L KIN, 1.0 ml/L PPM™, and 0.3 g/L charcoal. The trial was run with Hindu Kush as the control since previous experiments showed that the media was effective for this strain. Table 10 shows the results of these experiments, showing that the media worked well for all strains tested. In the Table SE refers to the standard error, a measure of variability within the treatment.

TABLE 10
Explant rooting results

| | |
|---|---|
| height | health |

| Genotype | week | average | SE | average | SE |
|---|---|---|---|---|---|
| Hindu Kush | 2 | 0.89 | 0.08 | 4.80 | 0.13 |
| | 3 | 1.33 | 0.19 | 4.60 | 0.16 |
| | 4 | 1.19 | 0.25 | 4.60 | 0.16 |
| Sour Kush | 2 | 0.83 | 0.05 | 4.90 | 0.10 |
| | 3 | 1.42 | 0.12 | 4.40 | 0.16 |
| | 4 | 1.37 | 0.19 | 4.50 | 0.17 |
| Skunk Haze | 2 | 1.02 | 0.05 | 4.80 | 0.13 |
| | 3 | 1.50 | 0.13 | 4.70 | 0.15 |
| | 4 | 1.18 | 0.15 | 4.70 | 0.15 |

### Example 6

### Oryzalin Treatment (Third Trial)

The trial was repeated with HKU and NOS with moderate concentrations of oryzalin (20, 40, and 60 μM), and 1 ml/L PPM™ (plant preservative mixture, Diagnovation Technologies) in all medias and solutions to reduce contamination. The initial analysis of these plants (as of two days after treatment) showed that many plants were showing browning, particularly those given the higher doses, but there were also several controls with this issue. The next oryzalin trial with moderate oryzalin concentrations had a better survival rate. Trials were started on three other genotypes as well. Initial results have shown that the optimal treatment (concentration and time) with oryzalin varies between genotypes. Table 11 provides the transformation efficiency of the oryzalin treatment of two C. sativa genotypes (Hindu Kush and Super Nordle). Some genotypes were more sensitive to the oryzalin treatment (e.g. Sour Kush), and needed a lower concentration. In some cases, a longer or shorter incubation time was required. For example, there were 2 Hindu Kush (HKU) tetraploids in this trial, but the ratio of mixoploids was higher, so Hindu Kush may need a longer incubation in the oryzalin solution.

TABLE 11
Transformation efficiency of oryzalin treatment
on two C. sativa genotypes (n = 8)

| | Oryzalin | | | |
|---|---|---|---|---|
| genotype | treatment | # survived | # mixoploids | # tetraploids |
| Hindu Kush | Control | 7 | 0 | 0 |
| | 20 | 5 | 4 | 0 |
| | 40 | 3 | 1 | 2 |
| | 60 | 2 | 2 | 0 |
| Super Nordle | Control | 7 | 0 | 0 |
| | 20 | 7 | 3 | 4 |
| | 40 | 4 | 2 | 2 |
| | 60 | 1 | 0 | 1 |

### Example 7

### Stomata Counting Method

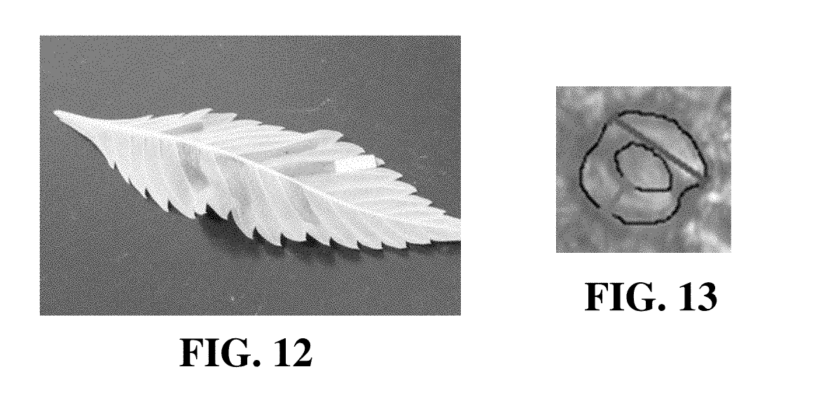

During the process of work with polyploidy, a method was identified for assessing the ploidy of a plant by counting stomata using a nail polish impression method. The Materials and equipment included Leaf samples, Scissors, Clear nail polish, Clear tape, Microscope slides, Compound microscope (Zeiss Lab.A1), Microscope camera (Axiocam 105 color camera), and a Laptop with imaging software (ZEN blue). The method was as follows: Several large fan leaves were collected from the plant being analyzed, a leaf sample was cut into individual leaflets and a thick coat of clear nail polish was applied to the underside of the leaf, between the veins. Once the first coat dried, another layer of the nail polish was applied. This process was repeated on several sections of the leaf and on various leaflets (more impressions were made than needed as they were not always successful). The leaves were set aside to dry overnight. FIG. 12 shows nail polish applied to several section of the underside of a C. sativa leaf. Then, once the leaves dried sections were cut out with the nail polish and a piece of clear tape was placed on top of the nail polish. The tape was crinkled so that the dried leaf material on the bottom of the nail polish impression fell off. The remaining material was gently brushed away (without using hard implements such as tweezers, as they will damage the impression). Using another piece of tape, the nail polish impression was stuck to a microscope slide so that the side where the nail polish was in contact with the leaf material was facing up. A coverslip was not used. Several impressions were prepared on a slide to ensure enough visible stomata. Under the 40× objective, sections of the impression were found where the entire field of view was clear. At least 5 photos of the impressions were taken for each plant/genotype being analyzed. Scale bars could be added in the ZEN program.

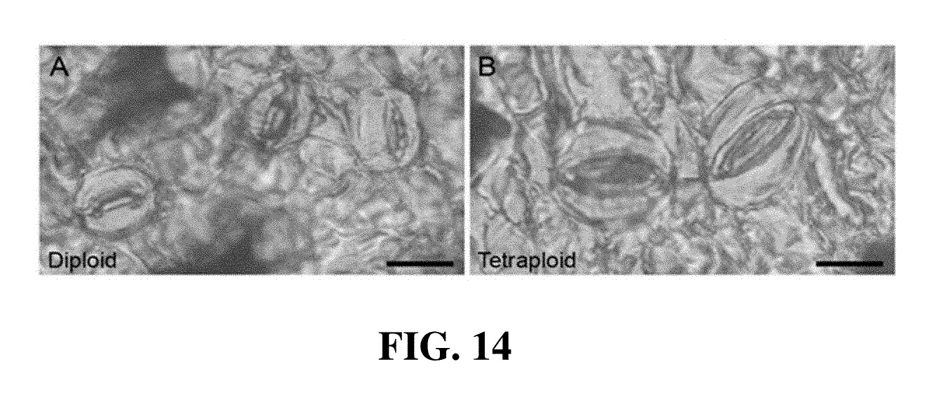

To assess stomatal density, the number of stomata was counted per field of view. Stomata at the edge of there image were only counted if they were more than halfway in the field of view (It may help to zoom in, and to mark the stomata as they are counted). The length and width of the guard cells were measured for at least 8 different stomata (measure the width of both guard cells). The line function in the ZEN program was used to measure the length and width directly on the image. FIG. 13 shows the measurements for the guard cells of C. sativa. FIG. 14 shows example of stomata from the underside of Super Nordle C. sativa plants. Panel A: diploid leaf, Panel B: tetraploid leaf. Images were taken at 400× magnification on a Zeiss Lab.A1 microscope with an Axiocam 105 color camera. The Data in Table 12 show the number of stomata, the guard cell length and the guard cell width and show correlation with diploidy or tetraploidy of the plants. For example, the number of stomata was about half in the tetraploid plant as compared to a diploid plants about 15 in the tetraploid and about 33 in the diploid. The results show that this method can be used as a quick method of assessing ploidy in addition to, or instead of, the root tip squash method and/or flow cytometry.

TABLE 12
Stomata characteristics on the underside of diploid
and tetraploid C. sativa plants

| | Diploid | | Tetraploid | | | |
|---|---|---|---|---|---|---|
| | Average | SE | Average | SE | P-value | Significant ? |
| # Stomata | 33.1 | 1.1 | 15.4 | 1.1 | 2.09e−8 | Y |
| Guard Cell | 32.0 | 1.1 | 43.3 | 1.0 | 0.0002141 | Y |
| Length | | | | | | |
| (μM) | | | | | | |
| Guard Cell Width | 9.1 | 0.3 | 11.7 | 0.2 | 0.0001091 | Y |
| (μM) | | | | | | |

### Example 8

### Oryzalin Treatment (Fourth Trial)

A further embodiment of the oryzalin treatment method was performed as follows: cuttings from mother plants were collected, axillary buds excised, and sterilized by soaking for 5 minutes in a 3% bleach solution with a drop of tween-20, then rinsing 3 times in sterile distilled water (each of these solutions was at approximately 4° C.). Four different genotypes were treated: Hindu Kush, Sour Kush, Skunk Haze, and Cannatonic. For oryzalin treatment, the axillary buds were placed in 50 mL falcon tubes with 20 mL of liquid MS media (30 g/L sucrose, 4.43 g/L MS basal salts). This solution was spiked with oryzalin (dissolved in 80% ethanol). Three concentrations were tested: Control: no oryzalin was added to the media, 20 μM oryzalin, and 40 μM oryzalin. Tubes with axillary buds and media were placed on a shake table (150 RPM) and covered in foil (to prevent the oryzalin from degrading in the light), and left to incubate in their solutions at room temperature. The Sour Kush, Skunk Haze, and Cannatonic were removed after 24 h. The Hindu Kush axillary buds were removed after 30 h. When the axillary buds were finished incubating in oryzalin, the oryzalin solution was poured off, and the axillary buds were rinsed three times with sterile water with 1 ml/L PPM. Treated axillary buds were placed in glass test tubes with shoot elongation media: 30 g/L sucrose, 4.43 g/L MS salts, 0.3 g/L charcoal, adjusted to pH 5.7, 8.0 g/L agar. Axillary bud cultures were moved to an incubator to grow at a temperature of approximately 24° C., and the light cycle was 16 hours of light. The height and explant health were monitored on a weekly basis. Table 13 shows the status of the explants after 10 days.

TABLE 13
Status of explants 10 days after oryzalin treatment (n = 10)

| | oryzalin | height | | health | |
|---|---|---|---|---|---|
| genotype | treatment | average | SE | average | SE |
| Cannatonic | control | 1.25 | 0.13 | 3.70 | 0.15 |
| | 20.00 | 0.77 | 0.08 | 3.30 | 0.21 |
| | 40.00 | 0.74 | 0.06 | 2.90 | 0.28 |
| Sour Kush | control | 1.11 | 0.13 | 3.60 | 0.16 |
| | 20.00 | 0.83 | 0.09 | 3.40 | 0.16 |
| | 40.00 | 0.63 | 0.07 | 3.22 | 0.14 |
| Skunk Haze | control | 0.84 | 0.09 | 3.60 | 0.16 |
| | 20.00 | 0.92 | 0.06 | 3.22 | 0.14 |
| | 40.00 | 0.63 | 0.07 | 3.10 | 0.18 |
| Hindu Kush | control | 0.87 | 0.20 | 3.30 | 0.15 |
| | 20.00 | 0.75 | 0.08 | 3.70 | 0.15 |
| | 40.00 | 0.78 | 0.09 | 3.50 | 0.17 |

At this point the axillary buds often lose their leaves and develop a deformed, globular meristem structure (It may take up to 2 months for shoots to develop from these structures). Once the explants have at least three leaves, a sample is taken to test on the flow cytometer to determine ploidy. The plants stay on shoot elongation media until the shoots are large and healthy enough to move to rooting. If an explant roots while on elongation media, it is left for an additional 2-3 weeks until the root system is well developed. For explants which are healthy but have not rooted yet, they are moved to the following rooting media: 30 g/L sucrose, 4.43 g/L MS salts, 0.3 g/L charcoal, adjusted to pH 4.7, 4.0 g/L Gelzan™, 1.0 mg/L IBA. Once on rooting media, it can take anywhere from 2-8 weeks for the roots to emerge. Once roots begin to grow, the cultures are left for an additional 2-3 weeks for the roots to develop. Once the cultures have a well developed root system, they are removed from culture and hardened off (method already discussed). From there, any tetraploids are cloned and analyzed for growth rate, yield, and chemistry.

### Example 9

### Super Nordle Tetraploid Analysis

Tetraploid clones were assessed for changes in phenotype or chemical profile compared to diploid control plants. While only minor changes were noted in the growth and chemistry of the tetraploids, this research lays important groundwork for the development of Cannabis strains with diverse chemical profiles, which would provide more options for medical and recreational Cannabis users. Using the following methods a tetraploid plant called “Super Nordle tetraploid” was produced and analyzed. The Super Nordle tetraploid was formed after treatment of C. sativa with a high concentration of oryzalin. The tetraploid was compared to a comparable diploid from the same strain. Mother plants for sampling were grown under 18 hours of light. Plants were watered daily with a nutrient solution (pH 5.5, EC 0.8 mS). Two strains were tested: one THC dominant indica strain (Hindu Kush), and one balanced THC/CBD indica dominant hybrid strain (Super Nordle). Nodal segments including young axillary buds with no fully expanded leaves were harvested from a healthy mother plant. Explants were taken from a single mother plant of each genotype to ensure consistency. Fan leaves and stipules were removed from the axillary bud, and the stem was cut at a 45° angle such that there was approximately 5 mm of stem below the axillary bud. The axillary buds were then sterilized in a 2% sodium hypochlorite solution (Old Dutch household bleach diluted with sterile distilled water, 0.1% v/v Tween-20) for 5 minutes, then rinsed three times in sterile distilled water for 1 minute each prior to inoculation on culture medium. Sterilized axillary bud explants were cultured in round-bottom glass culture vessels (25×150 mm test tubes with plastic caps, PhytoTechnology Laboratories C2093 and C1805) containing 20 mL of shooting media. The shooting media was composed of 1× Murashige & Skoog (MS) basal media with vitamins (PhytoTechnology Laboratories, M519) supplemented with 30 g/L sucrose (VWR SS1020), 0.3 g/L charcoal (PhytoTechnology Laboratories, A296), adjusted to pH 5.75 (±0.05), and solidified with 8.0 g/L agar. Plant growth regulators were added after autoclaving, 0.1 mg/L □-naphthaleneacetic acid (PhytoTechnology Laboratories, N600) and 0.4 mg/L kinetin (PhytoTechnology Laboratories, K750). Sterile shoots emerged after one to five months. Plantlets with elongated shoots (taller than 2.5 cm) were moved to larger glass vessels with vented caps (62×95 mm glass jar, PhytoTechnology Laboratories, C2099 and C176) containing 50 mL of rooting media. Rooting media was solidified with 4.0 g/L Gelzan™ (PhytoTechnology Laboratories, G3251) and contained 1.0 mg/L indole-3-butyric acid (PhytoTechnology Laboratories, 1538). Roots typically emerged after 3-5 weeks. If plantlets rooted in the shooting media they were not moved. All cultures were incubated at 24° C. with white fluorescent lights (16 hour photoperiod, average light intensity 75 μmol/m² s). Once plants had developed a strong root system (generally about 3 weeks after root emergence), they were removed from the media and transplanted into soil to harden off. The media around the soil was gently broken up, and remaining media was rinsed off the roots with lukewarm tap water. Plantlets were placed in 500 mL plastic pots with high porosity growing medium with mycorrhizae (Pro-Mix, Product 20381) and transferred to a temperature and humidity controlled growth room (24° C. and 40% relative humidity). Plants were grown under white fluorescent lighting (18 hour photoperiod; average light intensity 115 □mol m⁻² s⁻¹). The plantlets were kept under a humidity dome for the first week or two, venting the domes near the end to gradually bring down the humidity. Once the humidity domes were removed, the plants were watered daily with a fertilizer solution (General Hydroponics Cocotek Grow A/B, prepared to an electrical conductivity of 1.0 mS cm⁻¹). Disinfected axillary buds (10 replicates per genotype) were placed in treatment media containing 0 (control), 50, 100, or 150 □M oryzalin (3,5-dinitro-N⁴,N⁴-dipropylsulfanilamide) to induce polyploidy (PhytoTechnology Laboratories, 0630). A second trial was conducted using 0 (control), and 20, 40 or 60 □M oryzalin concentrations (8 replicates per genotype). The treatment media was prepared by diluting a stock solution (37.5 mM oryzalin in 80% ethanol) into 25 mL of liquid MS media (with 30 g/L sucrose, pH 5.75). A control was prepared with 100 μL of 80% ethanol (equivalent to the volume of oryzalin stock added to the 150 μM treatment). The cultures were covered in tin foil to prevent light degradation of the oryzalin, then rocked on an orbital shaker (150 rpm). After 24 hours, the oryzalin solution was removed, and axillary buds were rinsed three times with sterile distilled water containing 1 mL/L of the broad-spectrum biocide Plant Preservative Mixture (Plant Cell Technology). The axillary buds were then placed on shoot elongation media (shooting media) and placed into an incubator and grown according to the culture methods. Once the explants had recovered and grown at least three leaves, one leaf per plant was sampled for flow cytometric ploidy analysis. If an explant had developed more than one primary stem, one leaf from each branch was tested. Plants determined to be tetraploid were transplanted into soil and grown to maturity. Generally, fewer than half of the axillary buds survived, with the 100 and 150 μM treatments resulting in nearly 100% mortality. The majority of the plants which did survive had very small, curled leaves and highly deformed meristems which somewhat resembled callus tissue. These structures persisted for several weeks before recovering and developing small shoots. The shoots recovered slowly in culture but rooted normally, and growth began to speed up once they were transferred to soil for hardening. Most of the shoots which regenerated were transformed (87%), though only one tetraploid was obtained from Super Nordle.

Total nuclear DNA content was assessed by flow cytometry. Young leaves were gathered from healthy Cannabis mothers or culture plants and stored in damp paper towel on ice for up to 24 hours prior to analysis. All materials and samples were kept on ice throughout the sample preparation. Leaf samples of 0.5 cm² were macerated with a razor blade in a Petri dish containing 750 μL of ice-cold LB01 buffer (described by Dolezel et al. 1989) and the suspension was passed through a 30 □m mesh filter to isolate the nuclei (Celltrics). The filtrate was treated with 50 μL of 1 mg/ml RNAse and stained with 250 μL of 0.1 mg/mL propidium iodide for 30 minutes in the dark. The ploidy analysis was carried out with a Gallios flow cytometer (Beckman Coulter, Ontario, Canada), with method parameters 465 V and 120 seconds on the high flow setting capturing data for at least 1000 nuclei per sample.

Genome size of the plants was determined by co-chopping the Cannabis leaf tissue with a Raphanus sativa “Saxa” (2n=2X=16 chromosomes, 2C=1.11 pg) standard. Relative DNA content was determined using fluorescence area (585/42 nm detector) and fluorescence peak means, coefficients of variation, and nuclei numbers were measured using the flowPloidy package in R (Smith et al. 2018). Genome sizes were measured using the flowPloidy package in R (Smith et al., 2018). Genome sizes were measured on three non-consecutive days to ensure accuracy. The ploidy level of the diploid mother plant and in vitro polyploid plants was confirmed by chromosome count using root tip squashes. Young, healthy roots were harvested from Cannabis plants and rinsed with tap water to remove all traces of media. The roots were placed in an Eppendorf tube with water and pretreated with nitrous oxide for 1 hour in a custom-built pressurized chamber at 160 psi to accumulate metaphase cells (Andres and Kuraparthy, 2013). The roots were then fixed in a 3:1 ethanol:acetic acid mixture at room temperature for 24-48 hours. The root tips were digested in 1M HCl for 5 minutes at 60° C., and then rinsed with ice cold water three times. The root tip cells were then excised and macerated on a microscope slide following the squash method of Tsuchiya and Nakamura (1979), and stained with a drop of 2% acetocarmine. Cells were imaged using a Zeiss Lab A1 microscope with an Axiocam 105 color camera microscope and ZEN blue software, and processed in GIMP 2. Chromosomes were counted in at least 3 root tip cells per genotype. Ploidy of the transformed plants was re-tested several times to ensure stability. Root tip squashes also confirmed that the rooted tetraploid plants contained 4n=40 chromosomes. The majority of the tetraploid plants were stable over a 10 month period, and the ploidy was consistent between various branches.

The genome size of the Super Nordle tetraploid generated in trial 1 was determined to be 3.93 pg, almost exactly twice the genome size of the mother plant (1.97 pg). In one case, a plantlet initially appeared to be tetraploid, but was mixoploid in the second analysis. Growth parameters were measured for diploid and tetraploid clones to assess the effects of polyploidy. To generate material for the analysis, healthy plants in tissue culture were transferred to soil and grown into mother plants. Phenotypic analysis was carried out on both tetraploids and cultured diploids in order to determine altered characteristics in the tetraploids.

Fifteen cuttings from each mother were rooted in peat-based foam plugs with Stim Root #1 rooting powder (Plant Prod, Ontario). The clones were covered with a humidity dome and irrigated with a nutrient solution (General Hydroponics Cocotek Grow A/B, prepared to an electrical conductivity of 1.0 mS cm⁻¹) until roots were established. Most clones were successfully rooted after three weeks at which point the humidity domes were removed. Plants were grown under white fluorescent lighting with an average fluence of 115 μmol/m² s 18 hour photoperiod (half lighting in the early stage of clone rooting). To assess rooting speed, development of roots was checked three times a week and the date of first root emergence was noted. After five weeks, 9 or 10 healthy clones per genotype were transplanted for further phenotype analysis into one gallon pots with high porosity growing medium with mycorrhizae (Pro-Mix, Product 20381). Particularly tall clones had their lower stems trimmed and were buried deeper than the shorter ones, a common practice in cannabis cultivation and is intended to ensure uniform light intensity and water use. Plants were watered daily with a nutrient solution: General Hydroponics Cocotek Grow A/B during the vegetative phase and General Hydroponics Cocotek Bloom A/G during the flowering phase (both prepared to an electrical conductivity of 2.5 mS cm⁻¹). The plants were grown in an indoor cultivation facility. The plants were grown for 4 weeks in the vegetative growth phase (18 hour photoperiod, average light intensity of 220 μmol/m² s under metal halide lamps), and for 9 weeks in the flowering phase (12 hour photoperiod, average light intensity of 485 μmol/m²s under high pressure sodium lamps). In the second week of the vegetative phase, the apical portion of the plant was removed to leave only 6 lateral branches remaining (topping). At this stage, each of the cloned plants were retested using flow cytometry to confirm that they were all tetraploids. In the final week of vegetative growth, the plants were transplanted into two-gallon pots and moved to the flowering room to acclimatize to the higher light intensity before being exposed to the flowering light cycle. Following this switch, the plants were pruned as required to remove excess leaves and small stems to ensure adequate light penetration and air flow in the canopy to discourage pathogens (weeks 1, 3, and 4 of flowering). Growth parameters were measured once a week starting at the time of clone transplant to one-gallon pots. Specifically, plant height (from soil to the highest apical meristem), stem diameter (1 inch above the soil level), cumulative length of all primary lateral branches (measured from node to apical meristem), and the width of the central leaflets (at the widest point including teeth using three mature fan leaves per plant) were measured. Since growth slows during flowering, measurements were taken every two or three weeks. Plants were harvested after 9 weeks of flowering corresponding to 13 weeks of growth following clone transplant to one-gallon pots. Upon harvesting, the plants were weighed whole, and then separated into bud, leaf, and stem portions. Each of these portions was weighed individually. The bud and leaf samples were set on trays to dry in a climate-controlled room for one week. The bud samples were composed of equal portions of popcorn and cola buds—buds from the top or bottom of a stem, respectively. Leaf samples were composed of equal portions of fan leaves and sugar leaves—large vegetative leaves lacking trichomes, and the reduced leaves which grow in the influorescence, respectively. The dry weight of the buds was measured to determine final yield.

Stomata Characteristics—Nail polish impressions were used to compare the size and density of the stomata on the adaxial surface of diploid and tetraploid mature fan leaves (Grant and Vatnick, 2004). The impressions were dried overnight and then viewed under a Zeiss Lab.A1 compound microscope with an Axiocam 105 color camera. The number of stomata per field of view under the 40× objective was used to calculate the density of stomata in eight different images. In each image, the length and width of three stomata guard cells were measured using Zeiss ZEN blue imaging and analysis software. The size of the image was measured to calculate the number of stomata per mm². Final stomata images were processed using GIMP 2.

Trichome Density Measurements—Two weeks before the plants were harvested, trichome density was measured on diploid and tetraploid sugar leaves. Three large stems per plant were selected at random, and the 4^{th} leaf from the apex was harvested. Leaves were placed carefully in large tubes to avoid damaging the trichomes. The adaxial surface of the central leaflet was imaged at its widest point, under approximately 10× magnification using a camera lens attachment on a stereoscope (Zeiss Stemi DV4). A ruler was included in each photo to provide a scale. Using this scale, the stalked glandular trichomes were counted within a 16 mm² area of each leaf on one side of the midrib. For very small leaves, a 9 mm² area was used to calculate the trichome density.

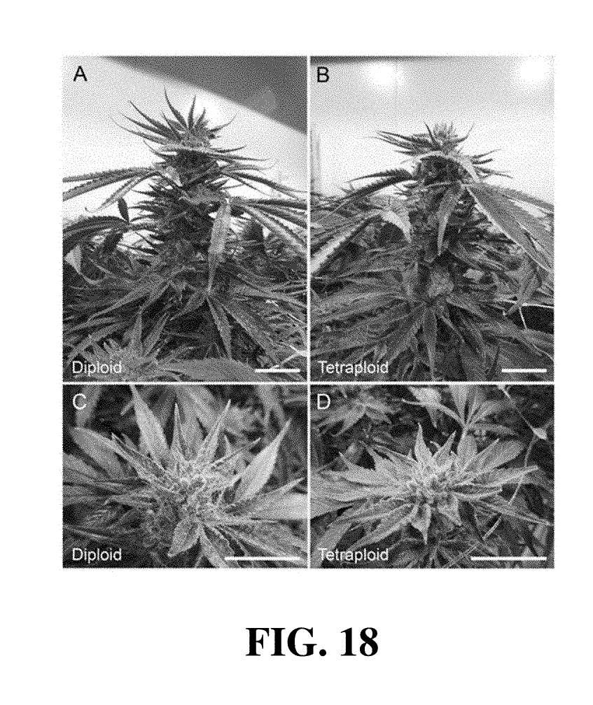

Chemotype Analysis of Diploid and Tetraploid Plants—Bud and leaf portions of diploid and tetraploid plants were sampled for analysis of cannabinoid and terpene content. For cannabinoid analysis, 0.5 g of dried tissue was homogenized and placed in a glass test tube with 10 mL of extraction solution (1:9 solution HPLC grade chloroform and methanol). The samples were then sonicated for 30 minutes and spun down. The extraction solution was filtered and diluted 10× in HPLC grade methanol. Cannabinoid samples were prepared in duplicate. For terpene analysis, 10 mg of homogenized sample was placed directly into a headspace vial. Twelve cannabinoids were assessed using an Agilent 1200 high performance liquid chromatograph (HPLC) with a diode array detector. Twenty three terpenes were assessed using Agilent 7820A/7890B gas chromatograph system with a flame ionization detector. Chemstation software (Open LAB CDS Chemstation Edition Rev. A.02.02(1.3)) was used to analyze the data. Peaks were identified using external cannabinoid and terpene standards. Final values are given as a percent (w/w) of the original dried material in FIG. 18. Final values in Tables 17 and 18 are given as milligrams of metabolite per gram of the original dried material. Data in all of the above tests was analyzed using unpaired Student's t-tests, or an unpaired two-sample Wilcoxon test in cases where data was not normally distributed. Analysis of variance with a Tukey's honest significant difference post hoc test was used to assess differences in phytochemical content. A chi-square test was used to compare rooting success. All tests were conducted at p<0.05 in the statistics program R (version 3.5.1). Graphs were plotted using Excel 2013.

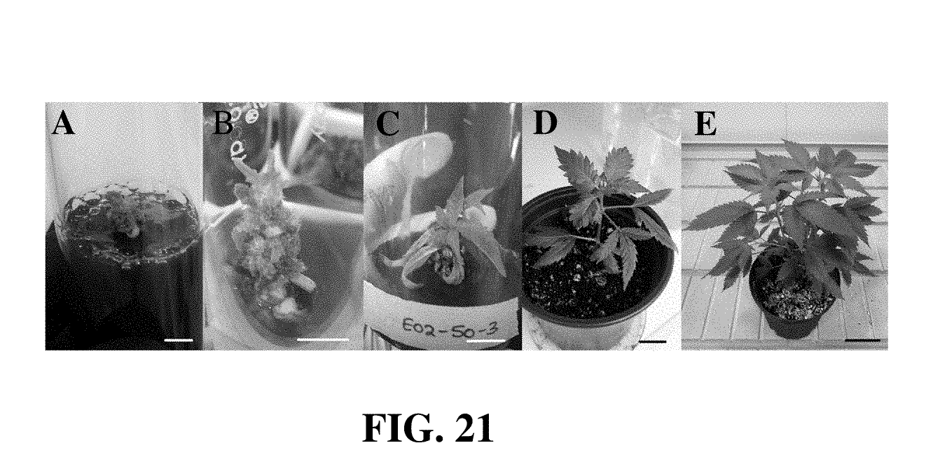

Survival Rate and Ploidy Determination—Oryzalin is a potent herbicide that inhibits microtubule polymerization (Morejohn et al., 1987). Axillary buds treated with high concentrations of oryzalin had a poor survival rate. No explants survived the 150 μM treatment. Survival rates for explants treated with 20 μM oryzalin ranged from 62% to 87.5% for Hindu Kush and Super Nordle, respectively (Table 14). The majority of surviving shoots had small, curled leaves and deformed meristems. These structures persisted for several weeks before recovering and initiating small shoots (FIG. 21). Flow cytometry analysis determined that nearly all the surviving shoots were successfully transformed (55% and 65% for Hindu Kush and Super Nordle, respectively). Of these, a large portion was mixoploid (69% and 47% for Hindu Kush and Super Nordle, respectively). Among the different treatments, 20 μM and 40 μM oryzalin had the best survival rates and produced the greatest number of tetraploids (Table 14). Overall, two tetraploid shoots were generated from Hindu Kush axillary buds and eight tetraploid shoots were generated from Super Nordle axillary buds. While Super Nordle tetraploid shoots recovered in culture and rooted normally, Hindu Kush tetraploid shoots grew poorly and failed to root. No further analysis was conducted on the Hindu Kush plants.

TABLE 14
Effect of oryzalin concentration on survival and polyploidization of C. sativa

| | Hindu Kush (High THC/Low CBD) | | | | Super Nordle (Balanced THC/CBD) | | | |
|---|---|---|---|---|---|---|---|---|
| Oryzalin | | Survival | Mixoploid | Tetraploid | | Survival | Mixoploid | |
| treatment | No. of | rate | plants | plants | No. of | rate | plants | Tetraploid |
| (μM) | explants | (%) | (%) | (%) | explants | (%) | (%) | plants (%) |
| 0 | 10 | 50 | 0 | 0 | 10 | 20 | 0 | 0 |
| 50 | 10 | 50 | 4 | 0 | 10 | 20 | 10 | 10 |
| 100 | 10 | 0 | 0 | 0 | 10 | 10 | 10 | 0 |
| 150 | 10 | 0 | 0 | 0 | 10 | 0 | 0 | 0 |
| 0 | 8 | 87.5 | 0 | 0 | 8 | 100 | 0 | 0 |
| 20 | 8 | 62.5 | 4 | 0 | 8 | 87.5 | 37.5 | 50 |
| 40 | 8 | 37.5 | 1 | 2 | 8 | 50 | 25 | 25 |
| 60 | 8 | 25 | 2 | 0 | 8 | 12.5 | 0 | 12.5 |

One representative Super Nordle tetraploid clone was selected for further analysis. Flow cytometry was used to determine a nuclear 2C DNA content of 3.93±0.23 pg (n=3) for the tetraploid, almost exactly twice the 1.97±0.04 pg (n=3) nuclear DNA content of the non-treated diploid mother plant (FIG. 9). The ploidy level of plants was confirmed by determining the chromosome number in root tip squashes. These data showed that tetraploid cells contained 2n=4x=40 chromosomes compared to 2n=2x=20 chromosomes in diploid cells. Note that x is the base number of chromosomes per genome and n is the number of chromosomes the individual has. The ploidy of the tetraploid clone and its progeny were assessed several times showing that ploidy was stable following transfer to soil and propagation through cuttings for phenotype analysis.

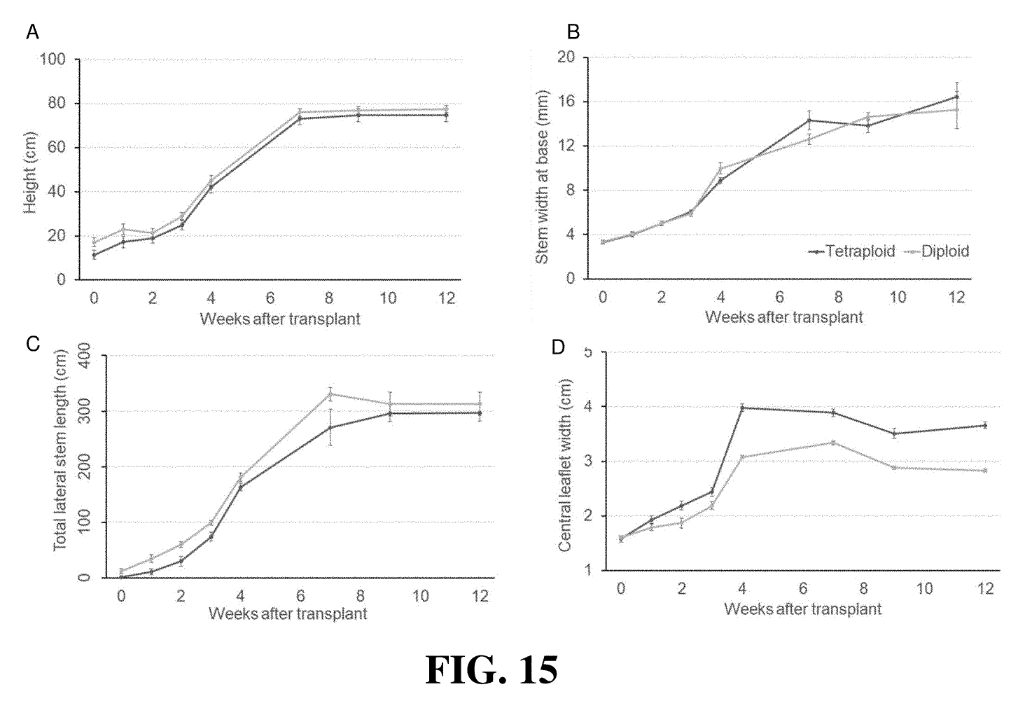

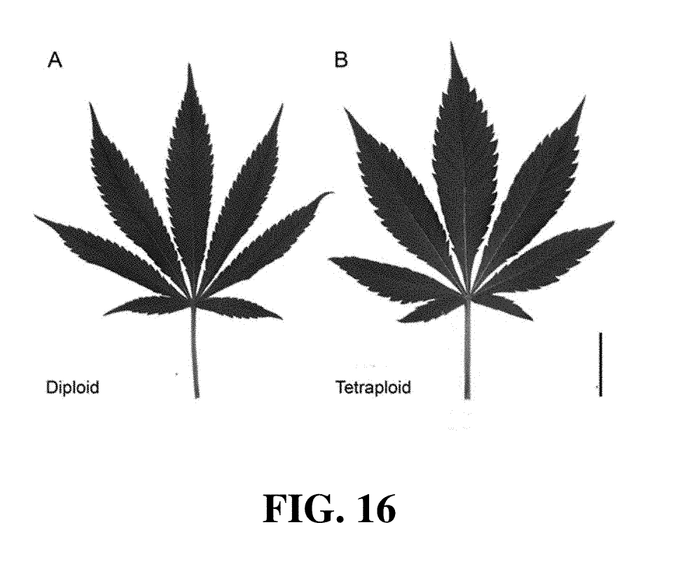

Tetraploid Phenotype—Significant effects of ploidy were noted on plant growth and morphology. To generate material for this analysis, diploid and tetraploid Super Nordle plants in tissue culture were transferred to soil and grown into mother plants. Fifteen cuttings per mother plant were rooted in soil for phenotypic assessment and chemical analysis. The polyploid strain showed a reduction in rooting success. After four weeks, only 66% of tetraploid clones were successfully rooted (n=9) compared to 100% of diploids (n=15). Among rooted tetraploids, root emergence was slightly delayed (16.0±3.7 days) compared to diploids (13.5±4.7 days). Ploidy effects on leaf morphology were also observed. Tetraploids had larger fan leaves compared to diploids (FIG. 16). The central leaflet was significantly wider by an average of 0.75 cm on tetraploid leaves compared to diploid leaves, during the flowering phase (FIG. 15). Nail polish impressions showed that stomata on the underside tetraploid fan leaves were about 30% larger and half as dense compared to diploids (Table 15 and FIG. 14).

TABLE 15
Stomata size and density (mean ± SE)

| | Stomatal Density | Guard Cell Length | Guard Cell Width |
|---|---|---|---|
| Ploidy | (mm²) | (□m) | (□m) |
| Diploid | 552.1 ± 18.2^{a} (n = 8) | 16.0 ± 0.5^{a} (n = 24) | 4.5 ± 0.1^{a} (n = 48) |
| Tetraploid | 256 ± 18.9^{b} (n = 8) | 21.7 ± 0.5^{b} (n = 24) | 5.9 ± 0.1^{b} (n = 48) |

Lower case letters indicate significant differences between measurements of a single metric (p < 0.05)

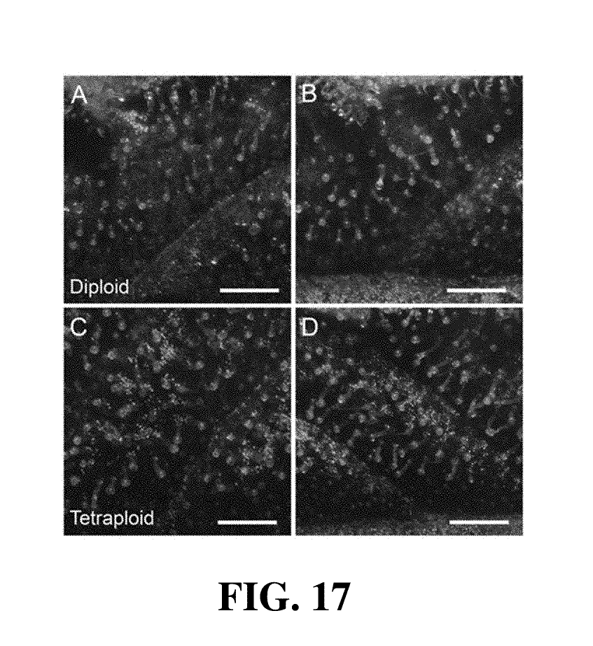

The height and stem base width of diploid and tetraploid plants were similar throughout growth. During the vegetative phase, tetraploid plants had slightly shorter lateral stems, but this difference was not significant following the switch to flowering (FIG. 15 Panels A, B, D). Plants of both ploidies showed their first flowers after one week under flowering lights, and the rate of floral growth was similar throughout the flowering phase. Trichome density on sugar leaves was measured at two weeks prior to harvest. Tetraploid leaves showed 40.4% higher glandular trichome density (4.41±0.16 trichomes per mm²) compared to diploids (3.14±0.15 trichomes per mm²). However, there was no obvious difference in the maturity of the trichomes on leaves, with the majority in the milky stage and some beginning to turn amber (FIG. 17).

The inflorescence apex and bud morphologies were similar for plants of both ploidies (FIG. 18). Tetraploid yields trended higher at harvest, but there was no significant difference in whole plant weight, weight of trimmed bud (buds trimmed of excess leaves) or trim weight (leaf trimmings) of diploids versus tetraploids (Table 16). Further, there no significant difference in the final dry weight of buds, which averaged 38.0±6.4 g per plant for tetraploids and 34.3±5.8 g per plant for diploids. These data indicate that chromosome doubling had no significant effect on plant growth, maturity, or yield.

TABLE 16
Yield metrics (mean ± SE) of Super Nordle C. sativa plants after 4 weeks of
vegetative growth and 8 weeks of flowering (n = 10 for diploids, n = 9 for tetraploids).

| | Weight (g) | | | |
|---|---|---|---|---|
| Ploidy | Whole plant | Wet bud | Leaf trim | Dry bud |
| Diploid | 527.78 ± 76.66 ^{a} | 134.50 ± 16.40 ^{a} | 145.60 ± 19.63 ^{a} | 34.35 ± 5.76 ^{a} |
| Tetraploid | 529.78 ± 99.22 ^{a} | 180.44 ± 30.90 ^{a} | 201.89 ± 37.95 ^{a} | 38.00 ± 6.37 ^{a} |

Lower case letters indicate significant differences between measurements of a single metric (p < 0.05)

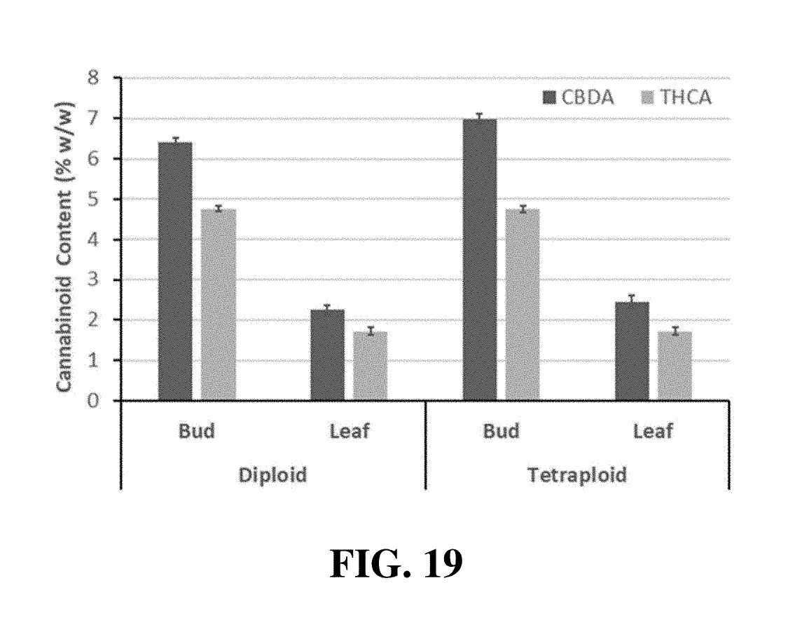

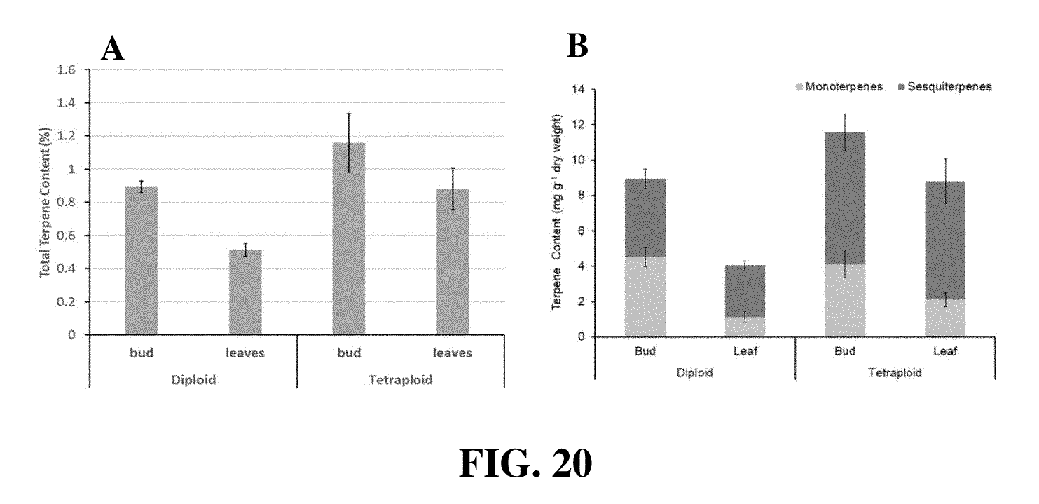

Phytochemical Content—THC and CBD are the main active ingredients in Cannabis, which in plants are mainly found in their acid forms (Andre et al., 2016). HPLC analysis showed that the ratio of THCA to CBDA was similar in Super Nordle diploids and tetraploids, with about 30% more THCA than CBDA (Table 17 and FIG. 19). Overall, the major cannabinoids comprised 64.16±0.98 mg g⁻¹ CBDA and 47.56±0.70 mg g⁻¹ THCA in the diploid buds, and 69.89±1.12 mg g⁻¹ CBDA and 47.56±0.76 mg g⁻¹ THCA in the tetraploid buds (Table 17). These values represent a significant 8.9% increase in CBDA in buds. No corresponding increase in THCA was found. Significant changes were also noted in the buds for some of the minor cannabinoids: a 34.3% reduction in cannabigerolic acid and a 15.2% increase in cannabidivarinic acid. No cannabinol, cannabicyclol, or Δ⁸-tetrahydrocannabinol (breakdown products) were detected in leaves or buds, and cannabidivarin was absent from the leaves. As expected, leaves had lower cannabinoid content, totaling about 35% the concentration of the buds (Table 17 and FIG. 19).

TABLE 17
Cannabinoid content (mean ± SE) in the dried leaf and bud material
of diploid and tetraploid Super Nordle C. sativa plants analyzed
in duplicate (n = 10 for diploids, n = 9 for tetraploids).

| | Content (mg/g dried tissue) | | | |
|---|---|---|---|---|
| Metabolite | diploid bud | diploid leaf | tetraploid bud | tetraploid leaf |
| Cannabidiol | 2.50 ± 0.10 ^{a} | 1.03 ± 0.04 ^{b} | 2.94 ± 0.15 ^{c} | 1.28 ± 0.07 ^{b} |
| Cannabidolic Acid | 64.16 ± 0.98 ^{a} | 22.46 ± 1.20 ^{b} | 69.89 ± 1.12 ^{c} | 24.58 ± 1.38 ^{b} |
| Δ⁹-tetrahydrocannabinol | 2.82 ± 0.09 ^{a} | 1.26 ± 0.05 ^{b} | 3.41 ± 0.12 ^{c} | 1.55 ± 0.08 ^{b} |
| Δ⁹-tetrahydrocannabinolic | 47.56 ± 0.70 ^{a} | 17.20 ± 0.92 ^{b} | 47.56 ± 0.76 ^{a} | 17.23 ± 1.01 ^{b} |
| acid | | | | |
| Cannabinol | 0 ^{a} | 0 ^{a} | 0 ^{a} | 0 ^{a} |
| Cannabigerol | 0.48 ± 0.01 ^{a} | 0.06 ± 0.02 ^{b} | 0.41 ± 0.01 ^{c} | 0.01 ± 0.01 ^{b} |
| Cannabigerolic acid | 1.46 ± 0.08 ^{a} | 0.33 ± 0.02 ^{b} | 0.96 ± 0.01 ^{c} | 0.28 ± 0.04 ^{b} |
| Δ⁸-tetrahydrocannabinol | 0 ^{a} | 0 ^{a} | 0 ^{a} | 0 ^{a} |
| Cannabichromene | 0.24 ± 0.07 ^{a} | 0 ^{b} | 0.12 ± 0.01 ^{ab} | 0.05 ± 0.03 ^{bc} |
| Cannabicyclol | 0 ^{a} | 0 ^{a} | 0 ^{a} | 0 ^{a} |
| Cannabidivarin | 0.01 ± 0.01 ^{a} | 0 ^{a} | 0.02 ± 0.01 ^{a} | 0 ^{a} |
| Cannabidivarinic acid | 0.33 ± 0.01 ^{a} | 0 ^{b} | 0.38 ± 0.01 ^{c} | 0 ^{b} |
| Total cannabinoids | 119.6 ± 1.81 ^{a} | 42.30 ± 2.22 ^{b} | 125.70 ± 2.10 ^{a} | 45.00 ± 2.50 ^{b} |

Lower case letters indicate significant differences between measurements of a single cannabinoid (p < 0.05)

More changes were apparent in the terpene profile of the tetraploids, which displayed an increase in total terpene content in the leaves. Terpenes that contribute to the taste and aroma of Cannabis products are mainly monoterpenes and sesquiterpenes (Andre et al., 2016). Tetraploids showed an increase in the overall terpene content of leaves (Table 18 and FIG. 18, Panel A). Total leaf terpenes were increased by 71.5% bringing the total terpene content to 8.8±1.26 mg g⁻¹ which was similar to the diploid buds. Tetraploid buds also had increased total terpene content, which reached 11.58±1.18 mg g⁻¹. Specific terpenes also showed significant changes. In buds and leaves, the monoterpene limonene was lower, whereas the sesquiterpene cis-nerolidol was increased, comprising up to 3.50 mg g⁻¹ in tetraploid buds. Overall, greater accumulation of sesquiterpenes was responsible for the increased terpene content of tetraploid leaves and buds (Table 18 and FIG. 18, Panel B). Tetraploid buds showed a 60% increase in guaiol. Tetraploid leaves also showed double the amount of sesquiterpene α-humulene and contained α-bisabolol, which was absent in the diploid leaves (Table 18).

TABLE 18
Terpene content (mean ± SE) in the dried leaf and bud material of diploid and tetraploid
Super Nordle C. sativa plants analyzed in duplicate (n = 10 for diploids, n = 9 for tetraploids).

| | | Content (mg/g dried tissue) | | | |
|---|---|---|---|---|---|
| Metabolite | Terpene class | diploid bud | diploid leaf | tetraploid bud | tetraploid leaf |
| α-Pinene | monoterpene | 1.06 ± 0.13 ^{a} | 0.51 ± 0.07 ^{b} | 1.03 ± 0.14 ^{a} | 0.56 ± 0.11 ^{b} |
| Camphene | monoterpene | 0 ^{a} | 0 ^{a} | 0 ^{a} | 0 ^{a} |
| β-Pinene | monoterpene | 0.51 ± 0.07 ^{a} | 0.21 ± 0.03 ^{b} | 0.41 ± 0.06 ^{a} | 0.20 ± 0.05 ^{b} |
| Myrcene | monoterpene | 2.29 ± 0.25 ^{a} | 1.11 ± 0.13 ^{bc} | 1.74 ± 0.23 ^{ab} | 0.87 ± 0.16 ^{c} |
| Δ-3-Carene | monoterpene | 0 ^{a} | 0 ^{a} | 0 ^{a} | 0 ^{a} |
| α-Terpinene | monoterpene | 0 ^{a} | 0 ^{a} | 0 ^{a} | 0 ^{a} |
| p-Cymene | monoterpene | 0 ^{a} | 0 ^{a} | 0.01 ± 0.01 ^{a} | 0 ^{a} |
| Limonene | monoterpene | 0.24 ± 0.06 ^{a} | 0.13 ± 0.04 ^{ab} | 0.06 ± 0.04 ^{b} | 0.01 ± 0.01 ^{b} |
| Eucalyptol | monoterpene | 0 ^{a} | 0 ^{a} | 0 ^{a} | 0 ^{a} |
| Ocimene | monoterpene | 0 ^{a} | 0 ^{a} | 0.05 ± 0.05 ^{a} | 0 ^{a} |
| γ-Terpinene | monoterpene | 0 ^{a} | 0 ^{a} | 0 ^{a} | 0 ^{a} |
| Terpinolene | monoterpene | 0 ^{a} | 0 ^{a} | 0.01 ± 0.01 ^{a} | 0 ^{a} |
| Linalool | monoterpene | 0.34 ± 0.03 ^{a} | 0.23 ± 0.03 ^{a} | 0.38 ± 0.09 ^{a} | 0.25 ± 0.04 ^{a} |
| Isopulegol | monoterpene | 0 ^{a} | 0 ^{a} | 0.12 ± 0.11 ^{a} | 0.03 ± 0.03 ^{a} |
| Geraniol | monoterpene | 0 ^{a} | 0 ^{a} | 0.27 ± 0.18 ^{a} | 0.15 ± 0.09 ^{a} |
| α-Terpineol | monoterpene | 0.08 ± 0.03 ^{a} | 0.06 ± 0.03 ^{a} | 0.03 ± 0.01 ^{a} | 0.01 ± 0.00 ^{a} |
| g-Terpineol | monoterpene | 0 ^{a} | 0 ^{a} | 0 ^{a} | 0 ^{a} |
| β- | sesquiterpene | 1.35 ± 0.06 ^{a} | 1.07 ± 0.06 ^{a} | 1.56 ± 0.19 ^{a} | 1.52 ± 0.24 ^{a} |
| Caryophyllene | | | | | |
| α-Humulene | sesquiterpene | 0.48 ± 0.03 ^{a} | 0.35 ± 0.04 ^{b} | 0.86 ± 0.20 ^{a} | 0.72 ± 0.12 ^{ab} |
| cis-Nerolidol | sesquiterpene | 2.12 ± 0.31 ^{ab} | 1.44 ± 0.24 ^{a} | 3.50 ± 0.41 ^{b} | 3.16 ± 0.51 ^{b} |
| trans-Nerolidol | sesquiterpene | 0 ^{a} | 0 ^{a} | 0.51 ± 0.38 ^{a} | 0.18 ± 0.18 ^{a} |
| Guaiol | sesquiterpene | 0.05 ± 0.01 ^{ab} | 0.04 ± 0.01 ^{a} | 0.08 ± 0.01 ^{b} | 0.07 ± 0.02 ^{ab} |
| α-Bisabolol | sesquiterpene | 0.41 ± 0.21 ^{ab} | 0 ^{a} | 0.97 ± 0.25 ^{b} | 1.04 ± 0.41 ^{b} |
| Total | | 4.52 ± 0.55 ^{a} | 1.14 ± 0.31 ^{b} | 4.10 ± 0.76 ^{ac} | 2.11 ± 0.38 ^{c} |
| monoterpenes | | | | | |
| Total | | 4.42 ± 0.55 ^{ab} | 2.89 ± 0.30 ^{a} | 7.47 ± 1.05 ^{b} | 6.70 ± 1.25 ^{b} |
| sesquiterpenes | | | | | |
| Total Terpenes | | 8.94 ± 0.36 ^{ab} | 5.13 ± 0.39 ^{a} | 11.58 ± 1.78 ^{b} | 8.80 ± 1.26 ^{b} |

Lower case letters indicate significant differences between measurements of a single terpene (p < 0.05)

As expected, the leaves had lower cannabinoid content—only about 35% the concentration of the buds. Terpene concentration was also lower in the leaves, though comparatively higher than the cannabinoids—76% for tetraploids and 57% for diploids. The treatment of axillary buds with oryzalin proved to be an effective method of inducing ploidy doubling in cannabis.

Past studies on the polyploidization of cannabis (and hops, the closest relative of cannabis), have used colchicine to induce tetraploids (Bagheri and Mansouri 2015, Roy et al. 2001, Trojak-Goluch and Skomra 2013). However, oryzalin is known to be more specific to plant tubulins; several authors have found that oryzalin is more effective and less toxic than colchicine, and that many of the side-effects of colchicine can be avoided by using oryzalin (Ascough et al. 2008, Stanys et al. 2006, Petersen et al. 2003, Dhooghe et al. 2009, Rego et al. 2011, Sakhanokho et al. 2009, Viehmannova et al. 2009). Trojak-Goluch and Skomra (2013) used a method of soaking explants similar to the one used here and found that 1250 μM of colchicine was the most effective for polyploidization of hops. The most successful oryzalin treatment in this case was about 40 μM, indicating that oryzalin is effective at over 30 times lower concentration.

Through these experiments, it was found that the optimal oryzalin treatment varies between genotypes. Hindu Kush tended to not tolerate the treatments as well as Super Nordle, and also yielded a much higher ratio of mixoploids. Subsequent experiments (unpublished data) have shown that a longer incubation time at lower concentration resulted in greater tetraploidization success in Hindu Kush. This suggests that this strain may have a longer cell cycle, since it requires a longer treatment time for all cells to have undergone replication. Similar differences in genotype response to oryzalin treatment have been found in other species such as cherry laurel and Japanese quince (Contreras and Meneghilli 2016, Stanys et al. 2006). In addition, the tetraploids recovered from Hindu Kush do not regenerate shoots easily on the current media. Compared to the Super Nordle tetraploids, these plants are sickly and slow growing. The majority of the Super Nordle tetraploids were stable over a period of 10-12 months. Of 10 total tetraploids generated in these two trials, one plantlet reverted to mixoploid status upon second analysis. Further testing will determine whether tetraploid stability lasts over multiple generations, and whether ploidy will be preserved if propagated through seeds, a result which was observed by Bagheri and Mansouri (2015) in hemp-type cannabis. Clone health and survival were poorer in the tetraploids, likely due to the significant decrease in rooting success. Previous studies in hops have found that tetraploids tend to have slower root development in culture, and have trouble acclimating to a greenhouse environment (Roy et al. 2001, Trojak-Goluch and Skomra 2013). However, surviving tetraploid clones grew and flowered at a rate comparable to diploids, yielding very similar amounts of dried bud. This contrasts results in other species which found tetraploids had either giant or dwarf phenotypes. For example, Chen and others (1979) found that tetraploid daylilies were significantly larger than diploids and had larger flowers. Conversely, Trojak-Goluch and Skomra (2013) found that tetraploid hops had shorter shoots and flowers than their diploid counterparts. Ploidy manipulation is a valuable tool in plant breeding. Important consequences of genome doubling can include larger organs and improved production of secondary metabolites, often linked to increased tolerance to biotic and abiotic stress. Polyploid forms also provide a wider germplasm base for breeding (Meru, 2012; Sattler et al., 2016). Polyploids have yet to be implemented in most breeding programs for Cannabis.

The results show that treatment of axillary buds with the dinitroaniline herbicide oryzalin is an effective method for chromosome doubling. Past studies on the polyploidization of hemp (Bagheri and Mansouri, 2015; Mansouri and Bagheri, 2017) and its closest relative hops (Humulus lupulus L.) used colchicine for doubling (Roy et al., 2001; Trojak-Goluch and Skomra, 2013). However, oryzalin has greater specificity for plant tubulins (Morejohn et al., 1987) and is considered as a more effective and less toxic alternative to colchicine (Petersen et al., 2003; Stanys et al., 2006; Ascough et al., 2008; Dhooghe et al., 2009; Sakhanokho et al., 2009; Viehmannová et al., 2009; Rêgo et al., 2011). Trojak-Goluch and Skomra (2013) found that 1250 μM of colchicine applied to explants was the most effective for polyploidization of hops. Shown here, concentrations in the range of 20 and 40 μM were the most effective for tetraploidization of Cannabis, indicating that oryzalin is effective at over 30 times lower concentration compared to colchicine. Hindu Kush was less tolerant of oryzalin treatment compared to Super Nordle and yielded a higher ratio of mixoploids. Similar genotype differences in response to oryzalin treatment have been found in other species such as cherry laurel and Japanese quince (Stanys et al., 2006; Contreras and Meneghelli, 2016).

A representative tetraploid was analyzed in this study. The ploidy of this strain proved stable through propagation in tissue culture and transfer to soil. Ploidy has also been stable throughout one generation of cloning. Seven subsequent Super Nordle tetraploids were isolated (Table 14). All of these plants have shown stable ploidy. An eighth potential tetraploid was isolated but reverted to mixoploid status upon second analysis. It is possible that this plant was initially mixoploid with a small portion of diploid cells that quickly multiplied (Blakeslee and Avery, 1937; Stanys et al., 2006). Overall, clone health and survival was lower among tetraploid clones, possibly due to lower rooting success. This finding matches with hops, whose tetraploids also have slower root development in culture and difficulty acclimating to a greenhouse environment (Roy et al., 2001; Trojak-Goluch and Skomra, 2013). Despite these early issues, tetraploid Super Nordle C. sativa plants grew and flowered at a rate comparable to diploids, yielding a similar amount of dried bud. Should this clone be representative, the data suggest that tetraploidization of Cannabis hinders rooting but has no significant negative effect on overall plant growth or yield.

A widespread consequence of polyploidy is an increase in cell size, caused by a larger number of gene copies. However, an increase in cell size does not always translate to increased size of the whole plant or its organ, since the number of cell divisions in polyploids can be reduced (Sattler et al., 2016). Measurements showed that the fan leaves of tetraploid Cannabis plants were larger than diploids, most evident during the flowering phase. Stomata were also about 30% larger (length and width) and less than half as dense (46%) compared to diploid leaves. Tetraploids of hemp also exhibited a lower density of stomata and stomata guard cells with larger length and diameter, but leaves were shorter and wider compared to diploids (Mansouri and Bagheri, 2017). Changes in stomata size and density are common among tetraploids (Ascough et al., 2008; Sakhanokho et al., 2009; Rêgo et al., 2011; Talebi et al., 2017). Overall, these data suggest that stomata size and density are reliable phenotypic markers for polyploid Cannabis.

The major cannabinoids THC and CBD in acid form are produced from a common cannabigerolic acid precursor by THCA synthase and CBDA synthase, respectively (Andre et al., 2016). The cannabinoid ratio is determined by codominant alleles of these synthase enzymes, which occur at a single locus on chromosome 6 (de Meijer et al., 2003; Marks et al., 2009). A number of allelic variants of these enzymes exist in different cultivars, and each has a unique effect on cannabinoid production. Therefore, large-scale genome rearrangements or duplications such as polyploidization could enable new allelic combinations, which have the potential to create novel chemotypes (Laverty et al., 2018).

Chemical analysis of Super Nordle tetraploids found little change in the cannabinoid profile relative to diploids. THCA content was similar and there was small but significant 8.9% increase of CBDA in tetraploid buds. The cannabigerolic acid precursor of cannabinoids is normally present at very low levels in the plant because of continual conversion to end products. Notably, tetraploids showed a 30% reduction in cannabigerol acid precursor. Linkage analysis suggests that availability of this precursor is a limiting factor in determining the overall yield of THC in plants (Laverty et al., 2018). Chemical analysis of tetraploid hemp found a 33% decrease in THC and little or no change in CBD content (Bagheri and Mansouri, 2015). These collective data suggest that ploidy may have limited influence on the cannabinoid biosynthetic pathway.

Terpenes are important aromatic compounds that determine the smell and taste of Cannabis products, and also modulate the drug effects of cannabinoids. Terpene concentrations above 0.5 mg g⁻¹ are considered pharmacologically relevant (Russo, 2011). In the buds and leaves, two additional sesquiterpenes reached this threshold in tetraploids, both of which have been found to be potent anti-inflammatories: □-humulene and □-bisabolol (Fernandes et al., 2007; Passos et al., 2007; Maurya et al., 2014). □-bisabolol is also known to be analgesic, antibiotic, and can moderately enhance skin penetration of other compounds (Kamatou and Viljoen, 2010). Additionally, although cis-nerolidol was above the biological relevance threshold in both diploids and tetraploids, this terpene was increased an average of 1.92-fold in the tetraploids. Nerolidol is a sedative and can interact with THC to enhance relaxation effects (Russo, 2011). This compound also functions as an excellent skin penetrant, which would be beneficial for topical Cannabis preparations (Kamatou and Viljoen, 2010). Although there was a decrease in limonene, this monoterpene is not present at concentrations likely to be biologically active.

Overall, total terpene content was increased in the leaves and buds of tetraploid Super Nordle plants. In general, terpene content was more variable in the tetraploids compared to diploids. This variability may be reflective of epigenetic instability which can occur in newly generated polyploids, resulting in greater variance between plants (Adams and Wendel, 2005; Comai, 2005). Sesquiterpenes were primarily responsible for the terpene increase in leaves and buds, suggesting a significant effect of ploidy on the cytosolic malvalonic acid biosynthetic pathway for sesquiterpenes. Monoterpenes, showing little change, come from a plastid-localized methyl-erythritol phosphate pathway whose geranyl diphosphate precursor is also a building block for cannabinoids (Flores-Sanchez and Verpoorte, 2008; Andre et al., 2016). A 71.5% increase in terpene content of leaves correlates well with increased trichome density on tetraploid sugar leaves. The terpene content of buds was also higher by about 30% suggesting that trichome density on flowers is also increased.

Although the phytochemical content is lower in leaves than in buds, particularly for the cannabinoids, this content is high enough for the trimmed leaf material to be used for extraction. Notably, the terpenes were increased in the tetraploid leaves to the point where the total terpene content was comparable to the diploid bud. Considering that the wet trim weight was usually similar to, or slightly higher than, the bud yield, extraction of quality trim material could almost double total production yield.

Results from this investigation, indicate that tetraploid Cannabis plants grow normally and have a similar chemical profile to diploids, with notable increases in CBD and sesquiterpenes. A key development in this study was the establishment of an efficient method of producing polyploids in Cannabis, laying the groundwork for larger scale production and assessment of tetraploids.

Any element of any embodiment may be used in any embodiment. Although the invention has been described with reference to specific embodiments, it will be understood by those skilled in the art that various changes may be made and equivalents may be substituted for elements thereof without departing from the true spirit and scope of the invention. In addition, modifications may be made without departing from the essential teachings of the invention. Identification of equivalent compositions, methods and kits are well within the skill of the ordinary practitioner and would require no more than routine experimentation, in light of the teachings of the present disclosure. Practice of the disclosure will be still more fully understood from the following examples, which are presented herein for illustration only and should not be construed as limiting the disclosure in any way.

All references cited in this specification, and their references, are incorporated by reference herein in their entirety where appropriate for teachings of additional or alternative details, features, and/or technical background.

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following claims.

### REFERENCES

Abdoli, M., Moieni, A., and Badi, H. N. (2013). Morphological, physiological, cytological and phytochemical studies in diploid and colchicine-induced tetraploid plants of Echinacea purpurea (L.). Acta physiologiae plantarum 35, 2075-2083. doi: 10.1007/s11738-013-1242-9
Adams, L., and Wendel, J. F. (2005). Polyploidy and genome evo plants. Current opinion in plant biology 8, 135-141. doi: 10.1016/j.pbi.2005.01.001
Andre, C. M., Hausman, J. F., and Guerriero, G. (2016). Cannabis sativa: the plant of the thousand and one molecules. Frontiers in plant science 7, 19. doi: 10.3389/fpls.2016.00019
Andres, R. J., and Kuraparthy, V. (2013). Development of an improved method of mitotic metaphase chromosome preparation compatible for fluorescence in situ hybridization in cotton. J. Cotton Sci. 17, 149-156.
Ascough, G. D., Van Staden. J., and Erwin, J. E. (2008). Effectiveness of colchicine and oryzalin at inducing polyploidy in Watsonia lepida NE Brown. HortScience 43, 2248-2251
Bagheri, M., and Mansouri, H. (2015). Effect of induced polyploidy on some biochemical parameters in Cannabis sativa L. Applied biochemistry and biotechnology 175, 2366-2375. doi: 10.1007/s12010-014-1435-8
Berkov, S., and Philipov, S. (2002). Alkaloid production in diploid and autotetraploid plants of Datura stramonium. Pharm. Biol. 40, 617-621. doi: 10.1076/phbi.40.8.617.14650
Blakeslee, A. F., and Avery, A. G. (1937). Methods of inducing doubling of chromosomes it plants: by treatment with colchicine. Journal of Heredity 28, 393-411. doi: 10.1093/oxfordjournals.jhered.a104294
Brenneisen, R. (2007). Chemistry and analysis of phytocannabinoids and other Cannabis constituents. In: Elsohly M (ed.). Marijuana and the Cannabinoids. Humana Press: Totowa, N.Y., pp. 17-49
Chaohua, C., Gonggu, Z., Lining, Z., Chunsheng, G., Qing, T., Jianhua, C., et al. (2016). A rapid shoot regeneration protocol from the cotyledons of hemp (Cannabis sativa L.). Industrial Crops and Products 83, 61-65. doi: 10.1016/j.indcrop.2015.12.035
Chen, C. H., & Goeden-Kallemeyn, Y. C. (1979). In vitro induction of tetraploid plants from colchicine-treated diploid daylily callus. Euphytica 28, 705-709. doi: 10.1007/BF00038937
Clarke, R. C. (1981). “Chapter 3: The Genetics and Breeding of Cannabis” in Marijuana botany: An advanced study: The propagation and breeding of distinctive cannabis. (Berkeley, Calif. Ronin publishing), 27-59. Retrieved from https://www.thevespiary.org/library/Files_Uploaded_by_Users/llamabox/Clarke,%20Robert%20C%20%20Marijuana%20Botany%20An%20Advanced%20Study.pdf on Aug. 29, 2018
Comai, L. (2005). The advantages and disadvantages of being polyploid. Nature reviews genetics 6, 836. doi:10.1038/nrg1711
Contreras, R. N., and Meneghelli, L. (201.6). In vitro chromosome doubling of Prunus laurocerasus ‘Otto Luyken’ and ‘Schipkaensis’. HortScience 51, 1463-1466. doi: 10.21273/HORTSCI11.1329-16
Corroon, J., and Phillips, J. A. (2018). A cross-sectional study of cannabidiol users. Cannabis and Cannabinoid Res. 3, 152-161. doi: 10.1089/can.2018.0006
De Meijer, E. P. M., Bagatta, M., Carboni, A., Crucitti, P., Moliterni, V. M. C., Ranalli, P., et al. (2003). The inheritance of chemical phenotype in Cannabis sativa L. Genetics 163, 335-346. doi: 10.1007/s10681-005-1164-8
De Moraes Pultrini, A., Galindo, L. A., and Costa, M. (2006). Effects of the essential oil from Citrus aurantium L. in experimental anxiety models in mice. Life Sciences 78, 1720-1725. doi: 10.1016/j.lfs.2005.08.004
Dermen, H. (1940). Colchicine polyploidy and technique. The botanical review 6, 599-635. doi: 10.1007/BF02919557
Dhooghe, E., Grunewald, W., Leus, L., and Van Labeke, M. C. (2009). In vitro polyploidisation of Helleborus species. Euphytica 165, 89-95. doi: 10.1007/s10681-008-9763-9
Divashuk, M. G., Alexandrov, O. S., Razumova, O. V., Kirov, I. V., and Karim, G. I. (2014). Molecular cytogenetic characterization of the dioecious Cannabis sativa with an XY chromosome sex determination system. PLoS One 9, c85118. doi: 10.1371/journal.pone.0085118
Doležel, J., Binarová, P., and Lucretti, S. (1989). Analysis of nuclear DNA content in plant cells by flow cytometry. Biologia plantarum 31, 113-120. doi: 10.1007/BF02907241
Doleźel, J., Sgorbati, S., and Lucretti, S. (1992). Comparison of three DNA fluorochromes for flow cytometric estimation of nuclear DNA content in plants. Physiol. Plant. 85, 625-631. doi: 10.1111/j.1399-3054.1992.tb04764.x
ElSohly, M. A. (2016) The botany of Cannabis sativa L. in The Analytical Chemistry of Cannabis (pp. 1-26). Amsterdam, Netherlands, Elsevier Inc. doi: 10.1016/B978-0-12-804646-3.00001-1
Fernandes, E. S., Passos, G. F., Medeiros, R., da Cunha, F. M., Ferreira, J., Campos, M. M. et al. (2007). Anti-inflammatory effects of compounds alpha-humulene and (−)-trans-caryophyllene isolated from the essential oil of Cordia verbenacea. European journal of pharmacology 569, 228-236. doi: 10.1016/j.ejphar.2007.04.059
Flores-Sanchez, I., and Verpoorte, R. (2008). Secondary metabolism in cannabis. Phytochem. Rev. 7, 615-639. doi: 10.1007/s11101-008-9094-4
Grant, B. W., and Vatnick, I. (2004). Environmental correlates of leaf stomata density. Teaching Issues and Experiments in Ecology. (Ecological Society of America: Washington, D.C.). Retrieved from https://tiee.esa.org/vol/v1/experiments/stomata/pdf/stomata.pdf in Oct. 23, 2018
Happyana, N. (2014) Metabolomics, Proteomics, and Transcriptomics of Cannabis sativa L. Trichomes. [dissertation/doctoral thesis][Germany] Technical University of Dortmund
Health Canada (2013). Information for Health Care Professionals: Cannabis (marihuana, marijuana) and the cannabinoids. Obtained from https://www.canada.ca/en/health-canada/services/drugs-medication/
Kamatou, G. P., and Viljoen, A. M. (2010). A review of the application and pharmacological properties of α-Bisabolol and α-Bisabolol-rich oils. Journal of the American oil chemists' society 87, 1-7. doi: 10.1007/s11746-009-1483-3
Lam, H. K., Harbard, J. L., & Koutoulis, A. (2014). Tetraploid Induction of Acacia crassicarpa using colchicine and oryzalin. Journal of Tropical Forest Science, 347-354.
Lata, H., Chandra, S., Techen, N., Khan, I. A., and ElSohly, M. A. (2016). In vitro mass propagation of Cannabis sativa L. A protocol refinement using novel aromatic cytokinin meta-topolin and the assessment of eco-physiological, biochemical and genetic fidelity of micropropagated plants. Journal of Applied Research on Medicinal and Aromatic Plants 3, 18-26. doi: 10.1016/j.jarmap.2015.12.001
Lata, H., Chandra, S., Khan, I., and ElSohly, M. A. (2009). Thidiazuron-induced high-frequency direct shoot organogenesis of Cannabis sativa L. In Vitro Cellular & Developmental Biology-Plant 45, 12-19. doi: 10.1007/s11627-008-9167-5
Laverty, K. U., Stout, J. M., Sullivan, M. J., Shah, H., Gill, N., Holbrook, L., et al. (2018). A physical and genetic map of Cannabis sativa identifies extensive rearrangement at the THC/CBD acid synthase locus Genome Res. 29, 1-11. doi: 10.1101/gr.242594.118
Majdi, M., Karimzadeh, G., Malboohi, M. A. Omidbaigi, R., and Mirzaghaderi, G. (2010). Induction of tetraploidy to feverfew (Tanacetum parthenium Schulz-Bip.): Morphological, physiological, cytological, and phytochemical changes. HortScience 45, 16-21
Mansouri, H., and Bagheri, M. (2017). “The induction of polyploidy and its effect on Cannabis sativa L.,” in Cannabis sativa L.—Botany and Biotechnology, eds. S. Chandra, H. Lata & M. A. Elsohly. (New York, N.Y. Springer International Publishing).
Marks, M. D., Tian, L., Wenger, J. P., Omburo, S. N., Soto-Fuentes, W., He, J., et al. (2009). Identification of candidate genes affecting Δ⁹-tetrahydrocannabinol biosynthesis in Cannabis sativa J. Exp. Bot. 60, 3715-3726. doi: 10.1093/jxb/erp210
Martin, S. L., Sauder, C. A., James, T., Cheung, K. W., Razeq, F. M., Kron, P., et al. (2015). Sexual hybridization between Capsella bursa-pastoris (L.) Medik (♀) and Camelina sativa (L.) Crantz (♂) (Brassicaceae). Plant Breed. 134, 212-220. doi: 10.1111/pbr.12245
Maurya, A., Singh, M., Dubey, V., Srivastava, S., Luqman S., and Bawankule, D. (2014). α-(−)-bisabolol reduces pro-inflammatory cytokine production and ameliorates skin inflammation. Current pharmaceutical biotechnology 15, 173-181
Mechoularn, R., Parker, A., and Gallily, R. (2002). Cannabidiol: an overview of some pharmacological aspects. The Journal of Clinical Pharmacology 42, 11S-19S. doi: 10.1002/j.1552-4604.2002.tb05998.x
Meru, G. M. (2012). “Polypoidy and its implications in plant breeding,” in Plant breeding in the 21st century, eds. C. Mcgregor & C. Brummer. (Athens, Ga. University of Georgia).
Mishra, B. K., Pathak, S., Sharma, A., Trivedi, P. K., and Shukla, S. (2010). Modulated gene expression in newly synthesized auto-tetraploid of Papaver somniferum L. S. Afr. J. Bot. 76, 447-452. doi: 10.1016/j.sajb.2010.02.090
Morejohn, L. C., Bureau, T. E., Molé-Bajer, J., Bajer, A. S., and Fosker, D. E. (1987). Oryzalin, a dinitroaniline herbicide, binds to plant tubulin and inhibits microtubule polymerization in vitro. Planta 172, 252-264.
Passos, G. F., Fernandes, E. S., da Cunha, F. M., Ferreira, J., Pianowski, L. F., Campos, M. M., and Calixto, J. B. (2007). Anti-inflammatory and anti-allergic properties of the essential oil and active compounds from Cordia verbenacea. Journal of ethnopharmacology 110, 323-333. doi: 10.1016/j.jep.2006.09.032
Petersen, K. K., Hagberg, P., and Kristiansen, K. (2003). Colchicine and oryzalin mediated chromosome doubling in different genotypes of Miscanthus sinensis. Plant Cell, Tissue and Organ Culture 73, 137-146. doi: 10.1023/A:1022854303371
Rêgo, M. D., Rêgo, E. R., Bruckner, C. H., Finger, F. L., and Otoni, W. C. (2011). In vitro induction of autotetraploids from diploid yellow passion fruit mediated by colchicine and oryzalin. Plant Cell, Tissue and Organ Culture 107, 451-459. doi: 10.1007/s11240-011-9995-6
Roy, A., Leggett, G., & Koutoulis, A. (2001). In vitro tetraploid induction and generation of tetraploids from mixoploids in hop (Humulus lupulus L.). Plant cell reports 20, 489-495. doi: 10.1007/s002990100364
Russo, E. B. (2011). Taming THC: potential cannabis synergy and phytocannabinoid-terpenoid entourage effects. British journal of pharmacology 163, 1344-1364. doi:10.1111/j.1476-5381.2011.01238.x
Sakhanokho, H. F., Rajasekaran, K., Kelley, R. Y., and Islam-Faridi, N. (2009). Induced polyploidy in diploid ornamental ginger (Hedychium muluense RM Smith) using colchicine and oryzalin. HortScience 44, 1809-1814
Sattler, M. C., Carvalho, C. R., and Clarindo, W. R. (2016). The polyploidy and its key role in plant breeding. Planta 243, 281-296. doi: 10.1007/s00425-015-2450-x
Smith, T. W., Kron, P., and Martin, S. L. (2018). flowPloidy: An R package for genome size and ploidy assessment of flow cytometry data. Applications in plant sciences 6. doi: 10.1002/aps3.1164
Stanys, V., Weckman, A., Staniene, G., and Duchovskis, P. (2006). In vitro induction of polyploidy in Japanese quince (Chaenomeles japonica). Plant Cell, Tissue and Organ Culture 84, 263-268. doi: 10.1007/s 11240-005-9029-3
Talebi, S. F., Saharkhiz, M. J., Kermani, M. J., Sharafi, Y., and Raouf Fard, F. (2017). Effect of different antimitotic agents on polyploid induction of anise hyssop (Agastache foeniculum L.). Caryologia 70, 184-193. doi: 10.1080/00087114.2017.1318502
Tavan, M., Mirjalili, M. H., and Karimzadeh, G. (2015). In vitro polyploidy induction: changes in morphological, anatomical and phytochemical characteristics of Thymus persicus (Lamiaceae). Plant Cell, Tissue and Organ Culture 122, 573-583. doi: 10.1007/s11240-015-0789-0
Thomas, B. F., and Elsohly, M. A. (2016). “The botany of Cannabis sativa L.,” in The Analytical Chemistry of Cannabis. (Amsterdam, Netherlands: Elsevier Inc.), 1-26.
Trojak-Goluch, A., and Skomra, U. (2013). Artificially induced polyploidization in Humulus lupulus L. and its effect on morphological and chemical traits. Breeding science 63, 393-399. doi: 10.1270/jsbbs.63.393
Tsuchiya, T., and Nakamura, C. (1979). Acetocarmine squash method for observing sugar beet chromosomes. Euphytica 28, 249-256. doi: 10.1007/BF00056582
Van Bakel, H., Stout, J. M., Cote, A. G., Tallon, C. M., Sharpe, A. G., Hughes, T. R., et al. (2011). The draft genome and transcriptome of Cannabis sativa. Genome Biol. 12, R102. doi: 10.1186/gb-2011-12-10-r102
Viehmannová, I., Cusimamani, E. F., Bechyne, M. Vyvadilová, M., & Greplová, M. (2009). In vitro induction of polyploidy in yacon (Smallanthus sonchifolius). Plant Cell, Tissue and Organ Culture 97, 21-25. doi: 10.1007/s11.240-008-9494-6
Wallaart, T. E., Pras, N., and Quax, W. (1999). Seasonal variation of artemis and its biosynthetic precursors in tetraploid Artemisia annua plants compared to the diploid wild-type. Planta Medica 65, 723-728. doi: 10.1055/s-1999-14094
Wilkinson, J. D., Whalley, B. J., Baker, D., Pryce, G., Constanti, A., Gibbons, S., and Williamson, E. M. (2003). Medicinal cannabis: is Δ9-tetrahydrocannabinol necessary for all its effects? Journal of Pharmacy and Pharmacology 55, 1687-1694. doi: 10.1211/0022357022304

1. A method for inducing polyploidy in a Cannabis plant, the method comprising treating the Cannabis plant or a part thereof with an amount of a dinitroaniline compound effective to induce polyploidy.

2. The method according to claim 1, wherein the Cannabis plant is Cannabis sativa.

3. The method according to claim 1, wherein the treatment is performed on a somatic tissue of the Cannabis plant.

4. The method according to claim 3, wherein the somatic tissue is an auxiliary bud.

5. The method according to claim 1, wherein the treatment comprises contacting the Cannabis plant or the part thereof with the amount of the dinitroaniline compound effective to induce polyploidy.

6. The method according to claim 1, wherein the dinitroalinine compound is selected from: benfluralin, butralin, chlornidine, dinitramine, dipropalin, ethalfluralin, fluchloralin, isopropalin, methalpropalin, nitralin, oryzalin, pendimethalin, prodiamine, profluralin, and trifluralin.

7. The method according to claim 1, wherein the dinitroaniline compound is 3,5-dinitro-N⁴,N⁴-dipropylsulfanilamide.

8. The method according to claim 1, wherein the dinitroalinine compound is oryzalin.

9. The method according to claim 1, wherein the amount of dinitroalinine compound effective to induce polyploidy is between about 5 μM and about 200 μM, about 10 μM and about 200 μM, about 50 μM and about 200 μM, about 5 μM and about 100 μM, about 10 μM and about 100 μM, about 20 μM and about 150 μM, about 20 μM and about 60 μM, about 50 μM and about 150 μM, or about 50 μM and about 100 μM.

10. The method according to claim 1, wherein the amount of dinitroalinine compound effective to induce polyploidy is about 5 μM, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about 100 μM, about 105 μM, about 110 μM, about 115 μM, about 120 μM, about 125 μM, about 130 μM, about 135 μM, about 140 μM, or about 145 μM

11. The method according to claim 1, wherein the amount of dinitroalinine compound effective to induce polyploidy is between about 5 μM and about 200 μM.

12. The method according to claim 1, wherein the amount of dinitroalinine compound effective to induce polyploidy is between about 20 μM and about 150 μM.

13. The method according to claim 1, wherein the amount of dinitroalinine compound effective to induce polyploidy is between about 20 μM and about 60 μM.

14. The method according to claim 1, wherein the treatment is performed for a time sufficient to induce polyploidy.

15. The method according to claim 14, wherein the period ranges from between about 12 hours and 48 hours.

16. The method according to claim 14, wherein the period ranges from between about 12 hours and 24 hours.

17. The method according to claim 1, wherein the treatment comprises contacting the Cannabis plant or the part thereof with the dinitoaniline compound.

18. The method according to claim 17, wherein the contacting comprises soaking the Cannabis plant or the part thereof in a composition comprising the dinitoaniline compound.

19. The method according to claim 1, further comprising obtaining a plantlet by culturing the treated Cannabis plant or the treated part thereof in a shoot elongation media for a time sufficient to induce rooting.

20. The method according to claim 1, wherein the treated Cannabis plant or the treated part thereof is placed to a rooting hormone comprising medium for a time sufficient to induce rooting.

21. The method according to claim 1, wherein the polyploidy is tetraploid.

22. A polyploid Cannabis plant or part thereof obtained by the method as defined in claim 1.

23. A Cannabis plant cell obtained by the method as defined in claim 1, wherein the Cannabis cell is polyploid.

24. A Cannabis plant cell obtained by the method as defined in claim 1, wherein the Cannabis cell is tetraploid.

25. A Cannabis plant cell according to claim 1, wherein the Cannabis cell is a Cannabis sativa cell.
